(19) United States
(12) Patent Application Publication
(10) Pub. No.: US 2023/0165521 A1
(43) Pub. Date: Jun. 1, 2023

(54) METHODS OF AND APPARATUS FOR ASSESSMENT OF SUBJECTS WITH DISORDERS OF CONSCIOUSNESS

(71) Applicants: Yeda Research and Development Co. Ltd., Rehovot (IL); Mor Research Applications Ltd., Ramat Gan (IL)

(72) Inventors: Noam SOBEL, Jaffa (IL); Anat ARZI, Rehovot (IL); Liron ROZENKRANTZ, Rehovot (IL); Lior GORODISKY, Rehovot (IL); Yael HOLTZMAN, Rehovot (IL); Yaron SACHER, Kochav Yair (IL); Danit ROZENKRANTZ, Tel Aviv-Yafo (IL)

(73) Assignees: Yeda Research and Development Co. Ltd., Rehovot (IL); Mor Research Applications Ltd., Ramat Gan (IL)

(21) Appl. No.: 17/919,267

(22) PCT Filed: Apr. 14, 2021
(86) PCT No.: PCT/IL2021/050429

§ 371 Date: Oct. 16, 2022

(30) Foreign Application Priority Data
Apr. 16, 2020 (IL) 273993

(51) Int. Cl. A61B 5/00; A61B 5/08; A61B 5/091

(52) U.S. Cl. CPC A61B 5/4842; A61B 5/091; A61B 5/0816; A61B 5/4011

(57) ABSTRACT

A method of assessing a subject including: measuring a plurality of respirations of the subject; determining one or more respiration parameter from the plurality of respirations; and determining, using the one or more respiration parameter, one or more of: a state of consciousness of the subject; and a prognosis of the subject.

### RELATED APPLICATION/S

This application claims the benefit of priority of Israel Patent Application No. 273993 filed on 16 Apr. 2020, the contents of which are incorporated herein by reference in their entirety.

### FIELD AND BACKGROUND OF THE INVENTION

The present invention, in some embodiments thereof, relates to assessment of patients and, more particularly, but not exclusively, to assessment of patients with disorders of consciousness (DoCs).

Background art, where each art is incorporated in its entirety by reference, includes the below list. In the following document these arts are referred to by number e.g. number in superscript.
1. Giacino, J. T. et al. Comprehensive systematic review update summary: Disorders of consciousness: Report of the Guideline Development, Dissemination, and Implementation Subcommittee of the American Academy of Neurology; The American Congress of Rehabilitation Medicine; And the. Neurology 91, 461-470 (2018).
2. Giacino, J. T., Fins, J. J., Laureys, S. & Schiff, N. D. Disorders of consciousness after acquired brain injury: The state of the science. Nat. Rev. Neurol. 10, 99-114 (2014).
3. Thibaut, A., Schiff, N., Giacino, J., Laureys, S. & Gosseries, O. Therapeutic interventions in subjects with prolonged disorders of consciousness. Lancet Neurol. 18, 600-614 (2019).
4. Stender, J. et al. Diagnostic precision of PET imaging and functional MRI in disorders of consciousness: A clinical validation study. Lancet 384, 514-522 (2014).
5. Schnakers, C. et al. Diagnostic accuracy of the vegetative and minimally conscious state: Clinical consensus versus standardized neurobehavioral assessment. BMC Neurol. 9, 1-5 (2009).
6. Price, J. L. Olfactory System. The Human Nervous System. Paxinos G (1990).
7. Merrick, C., Godwin, C. A., Geisler, M. W. & Morsella, E. The olfactory system as the gateway to the neural correlates of consciousness. Front. Psychol. 4, 1-15 (2014).
8. Mainland, J. & Sobel, N. The sniff is part of the olfactory percept. Chem. Senses 31, 181-196 (2006).
9. Wachowiak, M. All in a sniff: olfaction as a model for active sensing. Neuron 71, 962-73 (2011).
10. Frank, R. A., Dulay, M. F. & Gesteland, R. C. Assessment of the Sniff Magnitude Test as a clinical test of olfactory function. Physiol. Behav. 78, 195-204 (2003).
11. Johnson, B. N., Mainland, J. D. & Sobel, N. Rapid olfactory processing implicates subcortical control of an olfactomotor system. J. Neurophysiol. 90, 1084-1094 (2003).
12. Arzi, A. et al. Humans can learn new information during sleep. Nat. Neurosci. 15, 1460-1465 (2012).
13. Rozenkrantz, L. et al. A Mechanistic Link between Olfaction and Autism Spectrum Disorder. Curr. Biol. 25, 1904-1910 (2015).
14. Arzi, A., Rozenkrantz, L., Holtzman, Y., Secundo, L. & Sobel, N. Sniffing patterns uncover implicit memory for undetected odors. Curr. Biol. 24, R263-R264 (2014).
15. Reden, J., Draf, C., Frank, R. A. & Hummel, T. Comparison of clinical tests of olfactory function. Eur. Arch. Oto-Rhino-Laryngology 273, 927-931 (2016).
16. Rojas-Libano, D. & Kay, L. M. Interplay between Sniffing and Odorant Sorptive Properties in the Rat. J. Neurosci. 32, 15577-15589 (2012).
17. Johnson, B. N., Russell, C., Khan, R. M. & Sobel, N. A comparison of methods for sniff measurement concurrent with olfactory tasks in humans. Chem. Senses 31, 795-806 (2006).
18. Arzi, A. et al. Olfactory aversive conditioning during sleep reduces cigarette-smoking behavior. J. Neurosci. 34, 15382-15393 (2014).
19. Giacino, J. T., Ph, D., Kalmar, K. & Ph, D. COMA RECOVERY SCALE-REVISED ⓒ2004 Administration and Scoring Guidelines. (2004).
20. Rappaport, M., Dougherty, A. M. & Kelting, D. L. Evaluation of coma and vegetative states. Arch Phys Med Rehabil 73, 628-634 (1992).
21. Bareham, C. A. et al. Longitudinal bedside assessments of brain networks in disorders of consciousness: Case reports from the field. Front. Neurol. 9, (2018).
22. Wannez, S., Heine, L., Thonnard, M., Gosseries, O. & Laureys, S. The repetition of behavioral assessments in diagnosis of disorders of consciousness. Ann. Neurol. 81, 883-889 (2017).
23. Bareham, C. A. et al. Longitudinal assessments highlight long-term behavioural recovery in disorders of consciousness. Brain Commun. (2019). doi:10.1093/braincomms/fcz017
24. Doty, R. L. et al. Intranasal trigeminal stimulation from odorous volatiles: psychometric responses from anosmic and normal humans. Physiol. Behav. 20, 175-185 (1978).
25. Hummel, T. & Livermore, A. Intranasal chemosensory function of the trigeminal nerve and aspects of its relation to olfaction. Int. Arch. Occup. Environ. Health 75, 305-313 (2002).
26. Moor, J. W., Rafferty, A. & Sood, S. Can laryngectomees smell? Considerations regarding olfactory rehabilitation following total laryngectomy. J. Laryngol. Otol. 124, 361-365 (2010).
27. Green, P., Rohling, M. L., Iverson, G. L. & Gervais, R. O. Relationships between olfactory discrimination and head injury severity. Brain Inj. 17, 479-496 (2003).
28. Linacre, J. M., Heinemann, A. W., Wright, B. D., Granger, C. V. & Hamilton, B. B. The structure and stability of the functional independence measure. Arch. Phys. Med. Rehabil. 75, 127-132 (1994).
29. Rowe, T. B., Macrini, T. E. & Luo, Z.-X. Fossil Evidence on Origin of the Mammalian Brain. Science (80-. ). 332, 955 LP-957 (2011).
30. Sela, L. & Sobel, N. Human olfaction: A constant state of change-blindness. Exp. Brain Res. 205, 13-29 (2010).
31. Keller, A. Attention and olfactory consciousness. Front. Psychol. 2, 1-13 (2011).
32. Stevenson, R. J. Phenomenal and access consciousness in olfaction. Conscious. Cogn. 18, 1004-1017 (2009).
33. Nigri, A. et al. Central olfactory processing in subjects with disorders of consciousness. Eur. J. Neurol. 23, 605-612 (2016).
34. Arzi, A. et al. The influence of odorants on respiratory patterns in sleep. Chem. Senses 35, 31-40 (2009).
35. Arzi, A. et al. Olfactory aversive conditioning during sleep reduces cigarette-smoking behavior. J. Neurosci. 34, 5382-15393 (2014).
36. Forgacs, P. B. et al. Preservation of electroencephalographic organization in subjects with impaired consciousness and imaging-based evidence of command-following. Ann. Neurol. 16, 869-879 (2014).
37. Femandez-Espejo, D. et al. A role for the default mode network in the bases of disorders of consciousness. Ann. Neurol. 72, 335-343 (2012).
38. Noirhomme, Q., Brecheisen, R., Lesenfants, D., Antonopoulos, G. & Laureys, S. ‘Look at my classifier’s result’: Disentangling unresponsive from (minimally) conscious subjects. Neuroimage 145, 288-303 (2015).
39. Majdan, M. et al. Epidemiology of traumatic brain injuries in Europe: a cross-sectional analysis. Lancet Public Heal. 1, e76-e83 (2016).
40. Aidinoff, E. et al. Vegetative state outcomes improved over the last two decades. Brain Inj. 32, 297-302 (2018).
41. Monti, M. M. et al. Willful Modulation of Brain Activity in Disorders of Consciousness. N. Engl. J. Med. 362, 579-589 (2010).
42. Coleman, M. R. et al. Towards the routine use of brain imaging to aid the clinical diagnosis of disorders of consciousness. Brain 132, 2541-2552 (2009).
43. Napolitani, M. et al. Transcranial magnetic stimulation combined with high-density EEG in altered states of consciousness. Brain Inj. 28, 1180-1189 (2014).
44. Engemann, D. A. et al. Robust EEG-based cross-site and cross-protocol classification of states of consciousness. Brain 141, 3179-3192 (2018).
45. Bekinschtein, T. A. et al. Classical conditioning in the vegetative and minimally conscious state. Nat. Neurosci. 12, 1343-1349 (2009).
46. Sobel, N. et al. An impairment in sniffing contributes to the olfactory impairment in Parkinson’s disease. Proc. Natl. Acad. Sci. 98, 4154-4159 (2002).
47. Plotkin, A. et al. Sniffing enables communication and environmental control for the severely disabled. Proc. Natl. Acad. Sci. 107, 14413-14418 (2010).
48. Haviv, L. et al. Using a Sniff Controller to Self-Trigger Abdominal Functional Electrical Stimulation for Assisted Coughing Following Cervical Spinal Cord Lesions. IEEE Trans. Neural Syst. Rehabil. Eng. 25, 1461-1471 (2017).
49. Charland-Verville, V. et al. Detection of response to command using voluntary control of breathing in disorders of consciousness. Front. Hum. Neurosci. 8, 1-5 (2014).
50. Owen, A. Into the Gray Zone: A Neuroscientist Explores the Border Between Life and Death. (Simon and Schuster, 2017).
51. Borer-alafi, N., Sazbo, N. & Rn, C. K. O. Loewenstein communication scale for the minimally responsive subject. 16, (2002).
52. Seel, R. T. et al. Assessment scales for disorders of consciousness: Evidence-based recommendations for clinical practice and research. Arch. Phys. Med. Rehabil. 91, 1795-1813 (2010).
53. Rosenthal, R., Cooper, H. & Hedges, L. Parametric measures of effect size. Handb. Res. Synth. 621, 231-244 (1994).
54. Leech, N. L. & Onwuegbuzie, A. J. A Call for Greater Use of Nonparametric Statistics. Annu. Meet. Mid-South Edcational Res. Assoc. 1-24 (2002). doi:10.1016/1353-8292(95)00002-4
55. Kim, H.-Y. Statistical notes for clinical researchers: Chi-squared test and Fisher’s exact test. Restor. Dent. Endod. 42, 152 (2017).

### SUMMARY OF THE INVENTION

The present invention, in some embodiments thereof, relates to assessment of patients and, more particularly, but not exclusively, to assessment of patients with disorders of consciousness.

Following is a non-exclusive list including some examples of embodiments of the invention. The invention also includes embodiments which include fewer than all the features in an example and embodiments using features from multiple examples, also if not expressly listed below.

Example 1. A method of assessing a subject comprising:
measuring a plurality of respirations of said subject;
determining one or more respiration parameter from said plurality of respirations; and
determining, using said one or more respiration parameter, one or more of:
a state of consciousness of said subject; and
a prognosis of said subject.

Example 2. The method according to example 1, wherein said one or more respiration parameter includes a measure of variation of one or more feature of respiration of said subject, over said plurality of respirations.

Example 3. The method according to example 2, wherein one or more feature of respiration comprises duration of one or more of inhalation, exhalation, and of a single respiration.

Example 4. The method according to any one of examples 2-3, wherein said one or more feature of respiration includes an average value.

Example 5. The method according to any one of examples 1-2, wherein said determining comprises determining a state of consciousness of said subject, using one or both of:
a measure of variation of an inhale duration; and
a measure of variation of an exhale duration.

Example 6. The method according to example 3, wherein said determining comprises one or both of:
determining a probability that said subject is in a minimally conscious state; and
determining a probability that said subject is in a vegetative state.

Example 7. The method according to any one of examples 1-6, wherein said determining a prognosis comprises determining one or more of:
a probability of recovery of said subject; and
a probability of survival of said subject.

Example 8. The method according to example 7, wherein recovery includes transferring from a lower to a higher state of consciousness, over in a time period of one month to 1 year from respiration measurement.

Example 9. The method according to any one of examples 7-8, wherein survival is over a time period of 6 months to 3 years from respiration measurement.

Example 10. The method according to any one of examples 1-9 comprising:
performing an olfactory test on the subject; and
identifying presence or lack of a sniff response in response to said olfactory test, from subject nasal airflow measurements including at least one measured sniff volume;
wherein said one or more respiration parameter comprises said presence or lack of a sniff response; and
wherein said determining is using said presence or lack of a sniff response.

Example 11. A method of assessing a subject comprising:
measuring respiration of said subject;
performing an olfactory test on the subject; and
identifying presence or lack of a sniff response in response to said olfactory test, from subject nasal airflow measurements including at least one measured sniff volume; and determining, using said presence or lack of a sniff response, one or more of:
a state of consciousness of said subject; and
a prognosis of said subject.

Example 12. The method of example 11, wherein said performing comprises exposing said subject to an odor;
wherein said measuring comprises measuring nasal inhalation during said exposing to provide said at least one sniff volume and for a period of time not during said exposing to provide a volume of at least one baseline inhalation;
wherein said identifying comprises comparing said sniff volume with said baseline inhalation.

Example 13. The method of example 12, wherein said identifying comprises comparing a normalized sniff volume and a threshold, where normalized sniff volume is a ratio between said sniff volume and said baseline inhalation, to identify said presence or lack of said sniff response.

Example 14. The method according to example 13, wherein said threshold is 15%, where, if said normalized sniff volume exceeds said threshold, the patient is considered to have a sniff response.

Example 15. The method according to any one of examples 13-14, wherein said measuring is for a period of time providing a volume of a plurality of baseline inhalations, wherein said identifying comprises comparing said sniff volume with an average of said baseline inhalations.

Example 16. The method of example 15, wherein said period of time is immediately preceding said performing.

Example 17. The method according to any one of examples 15-16, wherein said identifying comprises excluding outlying baseline inhalations, from said plurality of baselines inhalations; wherein said average of said baseline inhalations is with included baseline inhalations.

Example 18. The method according to any one of examples 11-17, comprising:
repeating said performing and said identifying to provide a plurality of sniff volumes; and
determining a sniff volume variability from said sniff volumes;
comparing said sniff volume variability to a variability threshold to determine presence or lack of a sniff response.

Example 19. The method according to example 18, wherein said performing comprises presenting a single scent to said subject a plurality of times.

Example 20. The method according to example 18, wherein each said performing comprises presenting a first scent, or presenting a second scent, or performing a blank presentation.

Example 21. The method according to any one of examples 18-20, wherein said variability threshold is a difference in standard deviation of 0.35, wherein if said sniff volume variability is more than 0.35 across trials, the subject is considered to have a sniff response.

Example 22. The method according to any one of examples 10-21, wherein a sniff volume is excluded from said identifying is said sniff volume exceeds a threshold.

Example 23. The method of any one of example 10-22, comprising repeating said measuring, said performing and said identifying at intervals of time, to assess, over time one or more of:
a state of consciousness of said subject; and
a prognosis of said subject.

Example 24. The method of example 23, wherein said intervals of time are 1 day to 1 month.

Example 25. A system for assessing a subject comprising:
at least one sensor configured to sense respiration of said subject;
circuitry configured to:
   receive a measurement signal from said sensor;
   determine one or more respiration parameter from said measurement signal;
evaluate said subject, based on said one or more respiration parameter to provide an assessment of one or more of:
a state of consciousness of said subject; and
a prognosis of said subject.

Example 26. A system for assessing a subject comprising:
at least one sensor configured to sense respiration of said subject;
circuitry configured to:
   receive a measurement signal from said sensor;
   identify sniff inhalations from said measurement signal;
   evaluate said subject, based on said sniff inhalations to provide a patient evaluation.

Example 27. The system of example 26, wherein said at least one sensor is a spirometer.

Example 28. The system of any one of examples 26-27, wherein said sensor is fluidly attached to a cannula configured to be positioned within a subject’s nasal passageway.

Example 29. The system of any one of examples 26-28, wherein said measurement signal includes timing data of one or more olfactory test, wherein said circuitry is configured to identify sniff inhalations from said measurement signal using said timing data.

Example 30. The system according to any one of examples 26-29, wherein said circuitry is configured to identify baseline inhalations from said measurement signal.

Example 31. The system according to example 30, wherein said circuitry is configured:

to determine one or more averaged baseline inhalation volume; and
to normalize each sniff inhalation using an averaged baseline inhalation volume of said one or more averaged baseline inhalation volume to generate one or more normalized sniff inhalation.

Example 32. The system according to example 31, wherein said circuitry is configured to compare said one or more normalized sniff inhalation with a threshold to evaluate said subject.

Example 33. The system according to any one of examples 26-32, comprising a user interface; wherein said circuitry is configured to send said patient evaluation to said user interface for display to a user.

Example 34. A method of assessing a subject comprising:
measuring subject respiration;
performing an olfactory test on said subject;
identifying baseline inhalations and sniffing inhalations;
excluding outlying baseline inhalations and outlying sniffing inhalations; and
assessing said subject based on included baseline inhalations and included sniffing inhalations.

Example 35. The method of example 34, wherein said assessing comprises assessing said subject using a normalized sniff volume, which is determined by normalizing said included sniffing inhalations using an averaged baseline inhalation determined from said included baseline inhalations.

Example 36. The method of example 35, comprising comparing said normalized sniff volume with a threshold to assess said subject.

Example 37. The method according to any one of examples 34-36, comprising repeating said performing, said identifying, said excluding and said assessing to provide a plurality of normalized sniff volumes.

Example 38. The method of example 37, comprising excluding normalized sniff volumes of said plurality of normalized sniff volumes outside a threshold; and comparing a variability of included normalized sniff volumes with sniff volume variability threshold to assess said subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as assessing a subject, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a table illustrating theoretical respiration and sniff parameter relationships between different patient groups, according to some embodiments of the invention;

FIG. 1B is a method of assessing a subject, according to some embodiments of the invention;

FIG. 1C is a method of assessing a subject, according to some embodiments of the invention;

FIG. 2 is a system for assessing a subject, according to some embodiments of the invention;

FIG. 3 is a system for assessing a subject, according to some embodiments of the invention;

FIG. 4 is a simplified schematic illustration of an olfactory testing session, according to some embodiments of the invention;

FIG. 5A is a simplified schematic of nasal air flow with time before and during an olfactory test, according to some embodiments of the invention;

FIG. 5B is a simplified schematic of nasal air flow with time for a MCS subject, according to some embodiments of the invention;

FIG. 5C is a simplified schematic of nasal air flow with time for a VS/UWS subject, according to some embodiments of the invention;

FIG. 6A is a method of assessing a subject, according to some embodiments of the invention;

FIG. 6B is a method of assessing a subject, according to some embodiments of the invention;

FIG. 7A is a simplified schematic of a normalized sniff trace for olfactory tests, for a vegetative state/unresponsive wakefulness syndrome (VS/UWS) subject, according to some embodiments of the invention;

FIG. 7B is a simplified schematic of a normalized sniff trace for olfactory tests, for a minimally conscious state (MCS) subject, according to some embodiments of the invention;

FIG. 8A is a method of evaluating an odorant detection sniff response and/or a cognitively-driven sniff response of a subject, according to some embodiments of the invention;

FIG. 8B is a is a method of evaluating an odorant differentiation sniff response of a subject, according to some embodiments of the invention;

FIG. 9 is a method of processing olfactory testing data, according to some embodiments of the invention;

FIG. 10 is a method of olfactory testing, according to some embodiments of the invention;

FIG. 11 is a method of session assessment, according to some embodiments of the invention;

FIG. 12 is a method of determining an average baseline inhalation volume, according to some embodiments of the invention;

FIG. 13 is a method of assessing an olfactory trial, according to some embodiments of the invention;

FIG. 14 is a method of assessing sniff inclusion, according to some embodiments of the invention;

FIG. 15 is a simplified schematic of normalized sniff volume for different olfactory tests and different subject types, according to some embodiments of the invention;

FIG. 16 is a simplified schematic of normalized sniff volume for different subject types for successive sniffs after olfactory test presentation, according to some embodiments of the invention;

FIGS. 17A-C are simplified schematics showing results of different types of olfactory tests, for the first sniff after the trial, for VS/UWS subjects and MCS subjects, according to some embodiments of the invention;

FIGS. 18A-C are simplified schematics showing results of olfactory testing, for the first three sniffs after a trial, for VS/UWS subjects and MCS subjects, according to some embodiments of the invention;

FIGS. 19A-C are simplified schematics of sniff volume variability with sniff volume, for different olfactory tests, for VS/UWS subjects, according to some embodiments of the invention;

FIG. 20 is a simplified schematic of subject outcome and sniff response, for VS/UWS subjects, according to some embodiments of the invention;

FIGS. 21A-C are simplified schematics of sniff volume variability with sniff volume, for different olfactory tests, for DoC subjects, according to some embodiments of the invention;

FIG. 22 is a simplified schematic of subject outcome and sniff response, for DoC subjects, according to some embodiments of the invention;

FIGS. 23A-C are simplified schematics of Functional Independence Measure (FIM) score with normalized sniff volume, for different olfactory tests, according to some embodiments of the invention; and

FIG. 24 is a system for assessing a subject, according to some embodiments of the invention;

FIG. 25 is a simplified schematic trace 2500 of measurement of respiration, according to some embodiments of the invention;

FIG. 26 illustrates statistical significance of exemplary respiration parameters for subject assessment, according to some embodiments of the invention; and

FIG. 27 is a simplified schematic block diagram, according to some embodiments of the invention.

### DESCRIPTION OF SPECIFIC EMBODIMENTS OF THE INVENTION

The present invention, in some embodiments thereof, relates to assessing subjects and, more particularly, but not exclusively, to assessing state of consciousness and/or likelihood of recovery of subjects.

### Overview

A broad aspect of some embodiments of the invention relates to assessing an individual subject, based on one or more parameter of the subject’s measured respiration.

In some embodiments, the subject is suffering from a disorder of consciousness (DoC), for example, following brain injury.

In some embodiments, a respiration parameter characterizes one or both of inhalation and exhalation, for example, for a time period which is of longer duration than a single respiration. In some embodiments, a respiration parameter characterizes a subject’s response to a stimulation e.g. to olfactory stimulation.

In some embodiments, measured respiration parameters from measurement time duration/s when the patient is not intentionally submitted to stimulation are used.

In some embodiments, each respiration includes features including for example, the respiration trace itself, volume, duration, and peak airflow for each portion of the respiration, where portions include, for example, inhalation and exhalation.

In some embodiments, respiration parameters include average magnitude of respiration feature/s and/or variability of respiration feature/s over time. In some embodiments, higher average magnitude and/or higher variability of respiration parameters indicate that a subject is in better condition e.g. a higher state of consciousness and/or more likely to recover and/or survive.

Exemplary respiration parameters include, for a measurement time period, one or more of:
variability of the respiration measurement;
average and variability, for inhalation and/or exhalation, for one or more of; duration, peak airflow speed and volume.

In some embodiments, variability of the respiration measurement (also herein termed “trace”) is a respiration parameter. In some embodiments, this parameter, alone, is used to assess a subject. For example, to determine a likelihood that the subject will recover and/or survive.

In some embodiments, respiration parameters (e.g. including variability of the respiration) are determined using measurement of 1-100 respirations, or 10-100 respirations, or 50-100 respirations, 50-500 respirations, or 5000-20,000 respirations, or 1-30 minutes, or 1-20 mins, or 5-30 mins, or 30 mins-24 hours, or 2-8 hours, or about 6 hours, or about 24 hours.

Where, in some embodiments, the time period includes more than one full respiration. Where, in some embodiments, the time period is at least 1-20 mins, or 5-10 mins or lower or higher or intermediate durations or ranges.

In some embodiments, assessment includes determining which state of consciousness the subject is in e.g. generating a likelihood that the subject is in a particular state of consciousness, for one or more state of consciousness. In an exemplary embodiment, assessment includes determining whether the subject is in a minimally conscious state (MCS) or a vegetative state/unresponsive wakefulness syndrome state (VS/UWS).

In an exemplary embodiment, variability of duration for inhalation and/or exhalation is used to determine the consciousness state, for example, to indicate whether the individual is in a VS/UWS state or in a MCS. For example, in some embodiments, respiration duration variability above a threshold indicates that the patient is likely to be in a higher state of consciousness e.g. MCS as opposed to VS/UWS.

Alternatively or additionally to determine a state of consciousness, in some embodiments, a prognosis of an individual is predicted using measured respiration parameter/s of the individual. Where prognosis includes, recovery, for example, likelihood of transition to a higher state of consciousness (e.g. transition from VS/UWS to MCS to consciousness). Where, recovery, in some embodiments, is likelihood of recovering one or more function indicating of conscious awareness for example, the ability to perform visual tracking of a visual target (an object and/or a person). Where, recovery, in some embodiments, is likelihood of recovering one or more motor and/or cognitive skill. Where prognosis alternatively or additionally includes a likelihood of survival.

Where transition to a higher state of consciousness and/or survival are, for example, at a duration of over a month, or 1-3 months, or 1-6 months, over a year, or 1 month - 1 year, or 1 month - 3 years, or 1 month - 4 years, or 1-3 years, or 1-4 years, or lower or higher or intermediate time periods, after injury and/or after entrance into a disorder of consciousness state.

In an exemplary embodiment, one or more magnitude and/or variability, for one or more respiration parameter, are used to determine which VS/UWS patients will recover (e.g. to a MCS and/or to consciousness). Where higher respiration magnitude and/or respiration variability, in some embodiments, indicate increased likelihood of recovery.

In an exemplary embodiment, magnitude and/or variability for one or more respiration parameter, are used to determine which VS/UWS patients will recover (e.g. to a MCS and/or to consciousness). Where higher average respiration magnitude/s and/or higher respiration variability/ies, in some embodiments, indicate increased likelihood of survival.

Alternatively or additionally, in some embodiments, measured respiration is used to assess the patient when the patient is stimulated in one or more way, where different stimulations are provided at different times or sequentially. Where, in some embodiments, the subject is exposed to olfactory stimulation to provide sniff respiration parameters. For example, in some embodiments, one or more non-stimulated respiration parameter and/or one or more stimulated (e.g. olfactory simulation) respiration parameter are used to determine a patient state of consciousness e.g. probability of the subject being in a particular state and/or a patient prognosis e.g. probability of the patient surviving and/or transitioning from one state of consciousness to another.

An aspect of some embodiments of the invention relates to frequent monitoring and/or assessment of a subject using respiration parameters. Potentially, assessment of a subject using respiration measurements is more frequent (e.g. continuously) than clinical assessment/s. In some embodiments, monitoring of the subject using respiration parameters is used to determine when and/or how frequently a subject should be clinically evaluated e.g. when such an assessment would be of value. Potentially assessment of the subject using respiration measurements reduces the need for clinical assessment of the subject e.g. reducing the need to move the subject. In some embodiments, the respiration measurement and/or evaluation is performed in situ where the patient is cared for, potentially reducing and/or eliminating the need to move the patient for assessment and/or for invasive assessment/s.

In some embodiments, one or more respiration parameter is determined continuously e.g. using continuous respiration measurements. In some embodiments, the subject is assessed using these respiration parameter/s continuously. Alternatively or additionally, in some embodiments, continuous respiration measurements are used to assess the subject periodically.

In some embodiments, a subject is assessed using measured respiration parameters periodically. In some embodiments, respiration is measured (or continuous measurements are sampled) for short periods of time e.g. on a regular basis, for example, for 5-30 mins, 1-3 times a day.

In some embodiments, one or more respiration parameter is determined periodically. For example, sniff response or lack thereof, in some embodiments, is determined using periodic olfactory testing.

In some embodiments, assessment is performed using respiration parameters only (e.g. non-stimulated respiration and/or olfactory stimulation respiration parameter/s).

In some embodiments, for example along with respiration parameters (e.g. non-stimulated and/or olfactory stimulated) other measurement parameters are used in the assessment. For example, other physiological measurements e.g. blood pressure, temperature, cardiac measurement parameter/s.

In some embodiments, the assessment is used to provide information as to pain levels of the subject and/or effectiveness of medication (e.g. pain medication).

A broad aspect of some embodiments of the invention relates to assessing an individual subject, based on the subject’s measured inhalation in response to olfactory testing, also herein termed a subject’s “sniff”. In some embodiments, measured inhalation is nasal inhalation, e.g. from one or both nostrils. In some embodiments, assessment of the subject includes assessing whether sniff/s of the patient constitute a “sniff response” indicating that the subject has responded to the olfactory test/ing.

Without wanting to be bound by theory, it is theorized that sniff response of a subject indicates a level of corticothalamic integrity that is important for consciousness, and/or for life itself.

In some embodiments, despite variation in respiration volume and/or frequency between different patients and/or different patient groups, and/or for individual patients over time, sniffing is used to assess individual patients.

In some embodiments, alternatively or additionally to measured inhalation, exhalation/s are measured and, in some embodiments, are assessed to determine whether the subject has a sniff response. In some embodiments, one or more technique for determining sniff response from inhalation measurements is applied to exhalation measurements, for example, to determine a sniff response. For example, in some embodiments, measured exhalation is compared with a baseline exhalation (baseline exhalation, in some embodiments, determined using technique/s described elsewhere in this document but using inhalation measurements). For example, in some embodiments, a change from baseline of an exhalation of over a threshold percentage (e.g. 15%) is used to determine a sniff response.

In some embodiments, measured sniffs are used to assess a subject’s state of consciousness. In some embodiments, measured sniffs are used to assess a subject’s prognosis for example, likelihood of transfer from one state of consciousness to another, for example, likelihood of survival.

In some embodiments, subjects suffering from brain injury are assessed. In some embodiments, subjects which fail to inhale on command are assessed using measured inhalation in response to olfactory testing.

For example, in some embodiments, sniff response or lack thereof is used to determine a likelihood of a vegetative state/unresponsive wakefulness syndrome (VS/UWS) subject transferring to a minimally conscious state (MCS). For example, in some embodiments, a sniff response or lack thereof is used to determine a likelihood of a MCS subject recovering consciousness.

For example, in some embodiments, sniff response or lack thereof is used to determine a likelihood of mortality of a DoC subject.

In some embodiments, measured sniffs are used to assess one or more other type of assessment e.g. consciousness assessment. For example, in some embodiments, sniff response is used to re-assess a consciousness assessment of a subject. For example, in some embodiments, identification a sniff response in a VS/UWS subject is used to suggest that the VS/UWS subject has been misdiagnosed and should be classified as a MCS subject.

In some embodiments, feedback is used to improve subject assessment using sniff measurements. For example, in some embodiments, threshold/s are adjusted. For example, based on self-report from subjects of consciousness (e.g. when the subject was erroneously determined to lack consciousness) who later transition into consciousness.

A potential advantage of using sniffing to assess a subject is that, in some embodiments, a sniff response precedes other signs of recovery (e.g. consciousness recovery), for example, by days up to months.

In some embodiments, sniff response or lack thereof is used as a predictor of an extent of recovery possible for a subject. For example, prediction of a level of functional independence e.g. in the long term e.g. over month/s and/or year/s.

In some embodiments, olfactory testing includes making a presentation to a subject. In some embodiments, a presentation includes exposing a subject to an odorant.

In some embodiments, a single scent is used, potentially providing simple and/or rapid olfactory testing. For example, in some embodiments, normalized sniff volume for a single scent is used to assess a subject.

In some embodiments, more than one scent (e.g. pleasant and unpleasant scents) are used. In some embodiments, cognitive olfactory testing is performed. In some embodiments, olfactory testing includes exposing a subject to odorant/s multiple times e.g. in a single testing session.

In some embodiments, sniff measurement and/or assessment of a subject includes a first respiration (“first sniff after an olfactory trial. Alternatively or additionally, in some embodiments, measurement and/or assessment of the subject includes subsequent respiration/s after an olfactory trial (“second sniff”, “third sniff” etc.)

In some embodiments, a sensory-driven sniff response is identified, e.g. where a subject’s response to an scent e.g. a single scent is assessed.

In some embodiments one or both of two types of sensory-driven sniff responses are identified. For example, in some embodiments, an odorant detection sniff response is identified. For example, in some embodiments, an odorant differentiation sniff response is identified, e.g. where a difference in response to different odorants is assessed.

In some embodiments, a cognitive-driven sniff response is identified. For example, in an exemplary embodiment, subjects are told that they will be presented with odorants. If a subject then modifies nasal airflow in response to an cognitive olfactory test (e.g. a blank presentation), in some embodiments, this implies possible awareness of the presentation, and/or learned anticipation of an odorant.

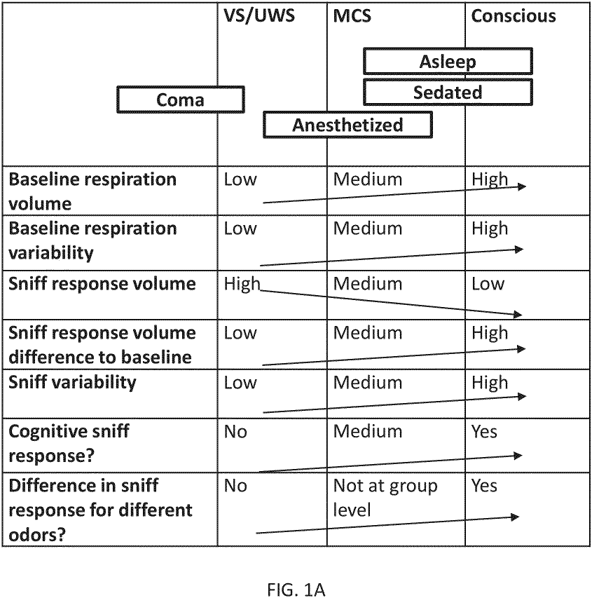

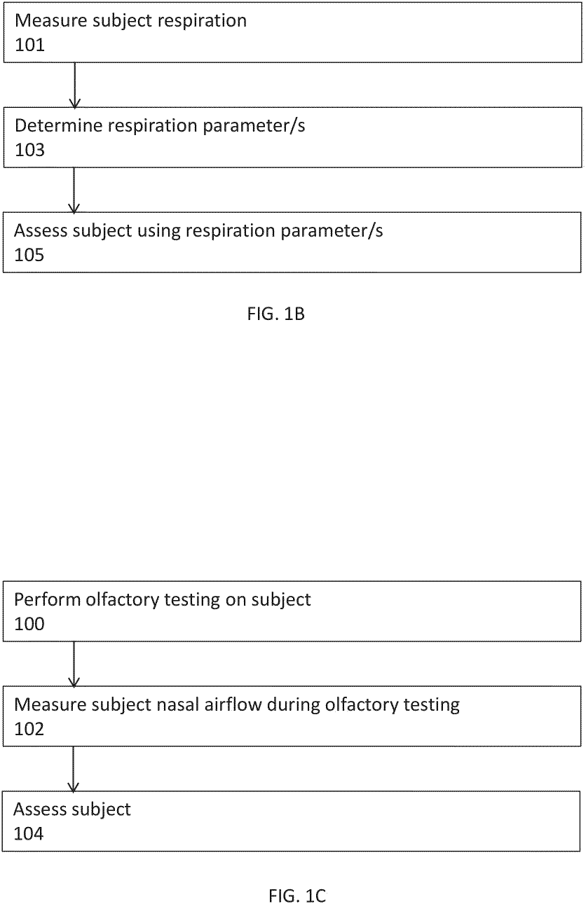

Without wanting to be bound by theory, FIG. 1A illustrates theoretical relationships between respiration parameters and different patient groups.

FIG. 1A is a table illustrating theoretical respiration and sniff parameter relationships between different patient groups, according to some embodiments of the invention.

FIG. 1A attempts to summarize and/or simplify respiration and sniff parameters for different groups of patients.

Experimental data in this document generally refer to three subject groups, DoC subjects in VS/UWS and MCS, and conscious subjects. However, in some embodiments, trends (e.g. illustrated on FIG. 1A with arrows) determined using these patient groups are used to assess other types of subject. For example, subjects in one or more of coma, anesthetized, sedated, asleep. Where exemplary position, in relation to other subject groups, for trends, is illustrated at the top of the table of FIG. 1A.

It is noted that, although most DoC subjects fail to inhale on command, but some DoC subjects which fail to inhale on command have a sniff response. Without wanting to be bound by theory, it is theorized that dissociation between volitional and odorant-induced sniffing, indicates the fundamental role of the olfactory brain in basic mechanisms of arousal.

Without wanting to be bound by theory, it is generally assumed that the more deeply unconscious a subject is, and/or the unhealthier the subject is (e.g. less likely to recover to a higher state of consciousness and/or to survive) the smaller the volume of respiration (e.g. one or both of inhalation and exhalation) and lower the complexity of the respiration (variability of the respiration and/or of one or more respiration feature and/or parameter).

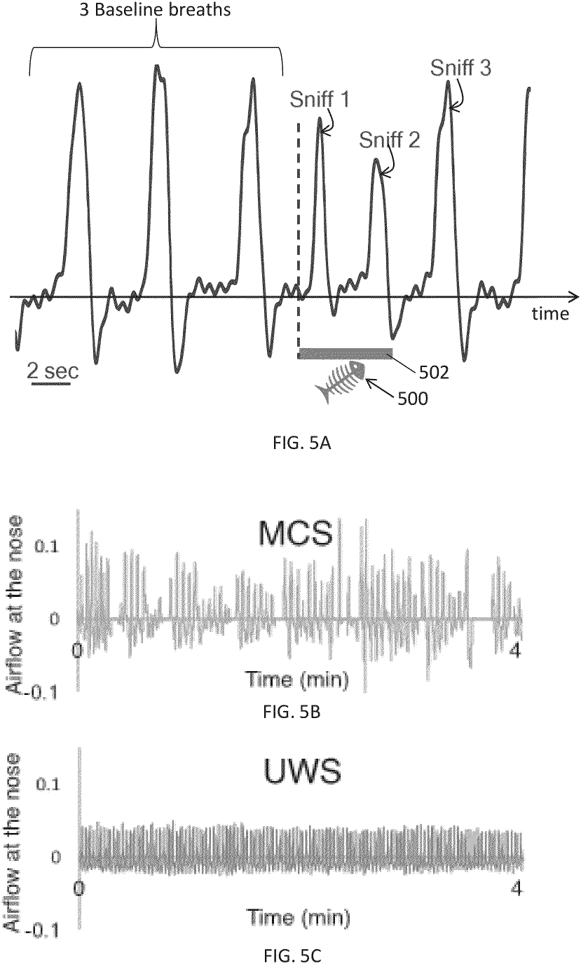

For example, in regard to “baseline respiration volume” and “baseline respiration variability” refer to FIG. 5B and FIG. 5C, showing exemplary traces for exemplary subjects where a higher level of both parameters, for the MCS subject measurement of FIG. 5B in comparison with the UWS subject measurement of FIG. 5C, is visually apparent.

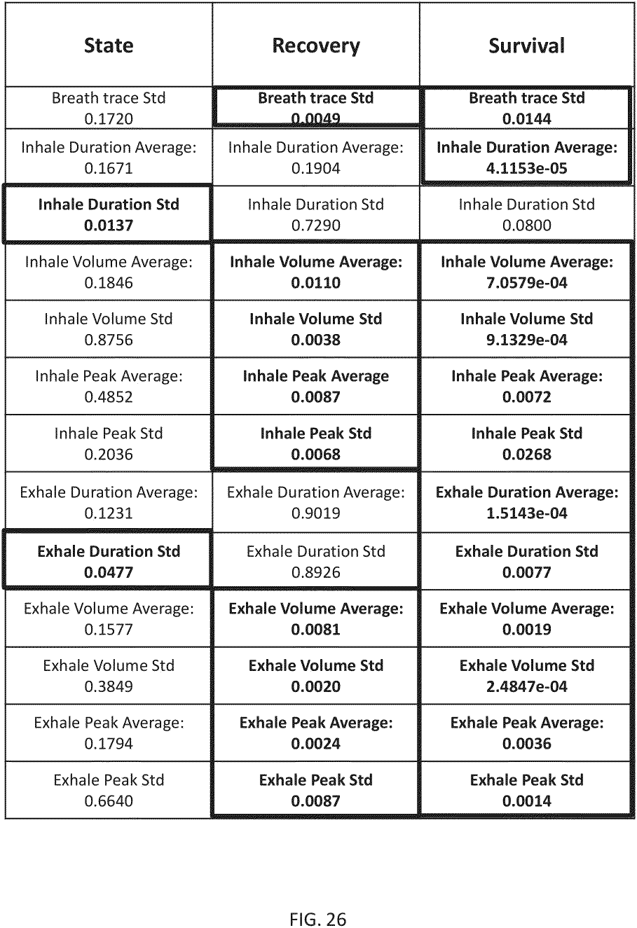

For example, referring to FIG. 26 which illustrates statistical significance of respiration parameters in differentiating between groups of subjects.

Without wanting to be bound by theory, it is generally assumed that the more deeply unconscious a subject is, and/or the unhealthier the subject is (e.g. less likely to recover to a higher state of consciousness and/or to survive) the smaller any response to olfactory stimulation is, and/or the lower the complexity of response to olfactory stimulation.

For example, the most unconscious subjects (and/or unhealthiest) are assumed to have no response to any kind of olfactory stimulation. With increasing consciousness (and/or health), it is assumed, in some embodiments, that responses to olfactory stimulation grow along with, in some embodiments, complexity of response.

For example, scent discrimination e.g. where a subject exhibits a different response to different scents, is considered to be a higher complexity sniff response, in some embodiments, than just a response to scent. For example, a cognitive sniff response, in some embodiments, is considered to be a higher complexity sniff response than just a response to scent.

The below examples of subject types and/or assessment are optionally related to this non-binding theory.

In some embodiments, one or more threshold for determining whether a subject has a sniff response is provided using an average from a subject group with higher consciousness levels. For example, in some embodiments, one or more threshold for VS/UWS subjects is based on average measurements from MCS subjects. E.g. in an exemplary embodiment, a sniff variability threshold for VS/UWS subject/s is based on average sniff variability measured in MCS subjects.

In some embodiments, one or more threshold is selected and/or adjusted and/or determined depending on a desired assessment e.g. of a patient and/or patient group. For example, one or more threshold is selected and/or adjusted and/or determined based on desired specificity and/or sensitivity. In some embodiments, a one or more threshold is selected and/or adjusted and/or determined based on relative importance (e.g. in the assessment) of specificity and/or sensitivity.

In some embodiments, MCS subjects are assessed using sniff differentiation. For example, in some embodiments, MCS subjects are assessed using sniff differentiation thresholds determined from measurements from conscious subjects. In some embodiments, MCS subject sniff differentiation is used to determine if and/or when a MCS subject will transition into full consciousness.

In some embodiments, a system for assessing subjects updates and/or generates thresholds e.g. as it collects and/or receives respiration parameter (e.g. sniff data) from different subjects.

In some embodiments, a level of coma of a patient is determined using a subject’s respiration parameter/s (e.g. non-stimulated respiration parameter/s and/or respiration response to olfactory testing).

In some embodiments, a depth of consciousness related to anesthesia is assessed using respiration parameter/s (e.g. non-stimulated respiration parameter/s and/or olfactory testing and sniff measurement). For example, in some embodiments, while a subject is under one or more type of general anesthetic, consciousness level of the subject is assessed using olfactory testing. Potentially, for example, assisting in identifying one or more of, when a subject should be intubated, when a subject is emerging for anesthesia (e.g. and should be given more anesthetic).

In some embodiments, respiration parameter/s (e.g. non-stimulated respiration parameter/s and/or olfactory testing and sniff measurement) are used to assess and/or predict recovery from anesthesia. For example, a duration of time for a subject to recover and regain full conscious awareness from anesthetic. For example, a severity of and/or a duration of short-term side effects (e.g. nausea and/or dizziness) and/or long-term side effects (e.g. impaired memory). For example, to detect onset of loss of consciousness following anesthetic administration and/or return of consciousness. For example, to detect when a subject has sensation of pain while under anesthetic e.g. during surgery from change/s in respiration pattern/s.

In some embodiments, a depth of sedation is assessed using respiration parameter/s (e.g. non-stimulated respiration parameter/s and/or respiration parameters from olfactory testing).

In some embodiments, respiration parameter/s (e.g. non-stimulated respiration parameter/s and/or respiration parameters from olfactory testing) is used in sleep assessment. For example to identify what stage of sleep a subject is in. For example, to assess a level of sensory and/or cognitive processing during different sleep stages, e.g. in comparison to wakefulness.

In some embodiments, the subject has respiration measurements collected and/or is subjected to olfactory testing e.g. to provide levels for comparison, before being anesthetized (and/or sedated and/or falling asleep).

In some embodiments, locked in syndrome is assessed and/or determined using olfactory testing.

In some embodiments, measured sniff volume/s are used to assess an individual subject. In some embodiments, a difference between measured sniff volume and non-stimulated respiration are used to assess the subject.

In some embodiments, sniffs are normalized using value/s associated with respiration levels without stimulation (e.g. without olfactory stimulation). Potentially mitigating effects of different volumes of respiration e.g. for an individual with time, and/or between patients and/or patient groups. In some embodiments, inhalation volumes are used in the normalization. Additionally or alternatively, in some embodiments, exhalation volume measurement/s are used e.g. to normalize e.g. using one or more feature as described in background reference number 34.

For example, in some embodiments, measured sniffing is used to assess subjects who have undergone tracheostomy. Where, some tracheostomy subjects, in some embodiments, exhibit sniff responses e.g. despite low volume of sniffs.

In some embodiments of the invention, threshold/s for assessing sniff parameter/s for an individual are provided using data from a patient group. In some embodiments, the patient group is a group in which the individual is classified (e.g. using clinical consciousness assessment/s). Alternatively or additionally, in some embodiments, threshold/s for assessing sniff parameter/s for an individual are provided using a data from a “healthier” and/or “more conscious” patient group.

For example, in some embodiments, sniffing measurements obtained from healthy awake individuals are used to identify sniff responses in subjects including non-healthy subjects e.g. those suffering from a disorder of consciousness (DoC) and/or subjects which are not fully conscious e.g. anesthetized, sedated, sleeping.

In an exemplary embodiment, a sniff volume threshold based on measurements from healthy awake subjects is used to assess measured sniffs for subjects with DoC.

In some embodiments, sniffing measurements from healthy awake individuals are used to define the parameter/s of what constitutes a sniff response. In an exemplary embodiment, a volume threshold for a sniff response obtained from olfactory testing in healthy awake individuals is used to identify sniff responses in other subjects.

In some embodiments, the volume threshold for assessment of sniffs is a ratio between non-stimulated inhalation and sniff inhalation e.g. to account for differences in respiration volume. In some embodiments, normalizing is using an averaged inhalation value, e.g. different averages for different types of patients.

In some embodiments, normalizing a sniff inhalation for an individual patient is using baseline inhalation of the individual patient. In some embodiments, an average value of baseline inhalation for the individual patient is used. In an exemplary embodiment, for a sniff inhalation, inhalations immediately preceding the sniff inhalation (e.g. an averaged number of) are used to normalize the sniff inhalation, for example, potentially reducing the effect of temporal variation in the subject’s inhalation. In some embodiments, outlying inhalations are excluded the baseline respiration average.

In some embodiments, inhalations are verified as being either sniff or baseline inhalations before calculating the normalized sniff volume. For example, using data which includes timing of olfactory presentations.

In some embodiments, alternatively or additionally to sniff volume, variability of sniff volumes are used to assess a subject. Where variability is determined using a plurality of sniff volumes e.g. as measured in an olfactory testing session. In some embodiments, variability of sniff volumes is compared with a threshold. The threshold, in some embodiments, is a patient group threshold. Use MCS for VS/UWS and use conscious for MCS.

A broad aspect of some embodiments of the invention relates to assessing a subject by selecting representative respiration values which are descriptive of the subjects state.

For example, in some embodiments, baseline respiration is filtered to remove outlying values (temporally outlying and/or outlying in magnitude) to provide averaged baseline respiration for normalizing sniff inhalation measurement.

For example, in some embodiments, outlying sniff inhalations are filtered to remove outlying values (temporally outlying and/or outlying in magnitude), before assessing a subject based on the sniff inhalation data.

In some embodiments, only baseline respiration measurements with sufficient temporal relationship to an olfactory test and/or sniff are used to normalize the sniff, e.g. as described, regarding step 1212FIG. 12.

In some embodiments, only sniff inhalations sufficiently temporally related to olfactory stimulation are used to assess the patient.

In some embodiments, filtering (e.g. of baseline inhalation/s and/or of sniff inhalation/s and/or of normalized sniff inhalation/s) is by comparison to an inclusion threshold where, in some embodiments, the inclusion threshold is a patient specific inclusion threshold. In some embodiments, an inclusion threshold is a patient group inclusion threshold. Where, in some embodiments, the patient group, e.g. in order to determine in inclusion threshold, is determined using clinical assessment techniques. In some embodiments, if filtering with a patient group inclusion threshold provides excess exclusions, the patient grouping of the patient is re-assessed.

In some embodiments, lack of a sniff response is used to assess a subject.

For example, in some embodiments, lack of a sniff response (e.g. in an individual categorized into a group where a sniff response is expected) is used to determine if a subject has damage to olfaction-related brain structure/s. For example, in some embodiments, an absence of a sniff response (e.g. in MCS subject/s) indicates a chronic or transient olfactory impairment.

A potential benefit of using respiration measurement e.g. including sniffing to assess a subject is the relative simplicity of equipment and/or testing technique/s required e.g. as opposed to imaging methods e.g. structural and/or functional brain imaging e.g. neuroimaging and electrophysiology.

A potential benefit of using respiration measurement e.g. including sniffing to assess a subject is that, in some embodiments, the assessment is performed at the subject’s bedside e.g. potentially being faster and/or less resource consuming and/or safer for delicate subjects.

In some embodiments, assessment of a subject using respiration parameter/s (e.g. non-stimulated respiration parameter/s and/or respiration parameters from olfactory testing) is performed repetitively over time, for example, to assess subject progression over time. For example, to assess effectiveness of treatment e.g. over time.

In some embodiments, respiration parameter/s monitoring (e.g. non-stimulated respiration parameter/s and/or olfactory testing) is used during and/or with treatment and/or stimulation of a subject, e.g. to assess effectiveness of the treatment and/or stimulation.

In some embodiments, respiration parameter/s (e.g. non-stimulated respiration parameter/s and/or sniff measurements) are used to select treatment and/or stimulation. For example, in some embodiments, a stimulation (for example aural e.g. music and/or visual e.g. video of loved ones) and/or treatment (e.g. medication given) is performed on a subject while and/or temporally close to olfactory testing sniff assessment, the stimulation then being assessed using the sniff measurements. In some embodiments, personalized treatment and/or stimulation plans are built using olfactory testing sniff assessment.

In some embodiments, treatment and/or stimulation is automated e.g. along with respiration measurement and/or olfactory testing and/or sniff assessment. For example, where measured feedback provided by sniff assessment is used to automatically trigger treatment and/or stimulation.

In some embodiments, respiration parameter monitoring and/or sniff assessment are performed repetitively, e.g. to provide a log of patient progress, optionally along with stimulation and/or treatment data.

In some embodiments, subjects who are ventilated (e.g. subjects in a coma, e.g. intubated anesthetized patients) are assessed using sniff assessment. For example, in some embodiments, sniffing is measured in-between ventilations and/or when ventilation is paused. For example, in some embodiments, where a patient is partially ventilated (e.g. using pressure support ventilation) changes in respiratory rate and/or tidal volume are used to identify a sniff response.

In some embodiments, muscles tone (EMG) in the face in response to odors is measured and used to identify a sniff response e.g. instead of sniffing for ventilated subjects, e.g. in addition and/or alternatively to respiration measurements.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Methods of Assessing Subjects

FIG. 1B is a method of assessing a subject, according to some embodiments of the invention.

At 101, in some embodiments, the subject’s respiration is measured. For example for a time period.

In some embodiments, respiration is measured e.g. by one or more sensor (e.g. sensor/s 104FIG. 1D sensor/s 204FIG. 2, sensor/s 304FIG. 3).

In an exemplary embodiment, nasal respiration is measured e.g. using a catheter inserted into a nostril e.g. as described elsewhere in this document.

At 103, in some embodiments, one or more respiration parameter is determined from collected respiration measurements. Where respiration parameters include, in some embodiments, averages and/or variability of one or more respiration feature. Where respiration features are, for example, as illustrated and/or described regarding FIG. 25.

In some embodiments, one or more respiration parameter is determined by respiration of the subject associated with stimulation of the subject. For example, olfactory stimulation of the subject for example, olfactory testing as described elsewhere in this document. Where, in some embodiments, respiration parameter/s include presence or lack of a sniff response.

At 105, in some embodiments, the subject is assessed using the respiration parameters determined at step 103. For example, where assessment includes determining a likelihood that the subject is in one or more particular state of consciousness and/or a likelihood of recovery and/or survival of the subject.

FIG. 1C is a method of assessing a subject, according to some embodiments of the invention.

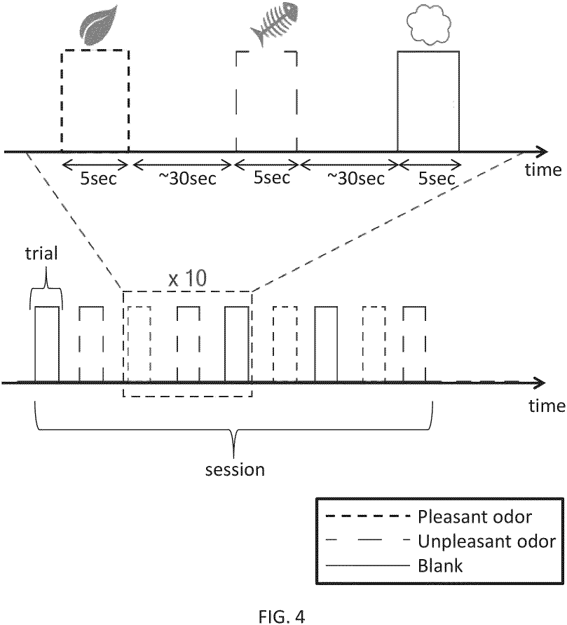

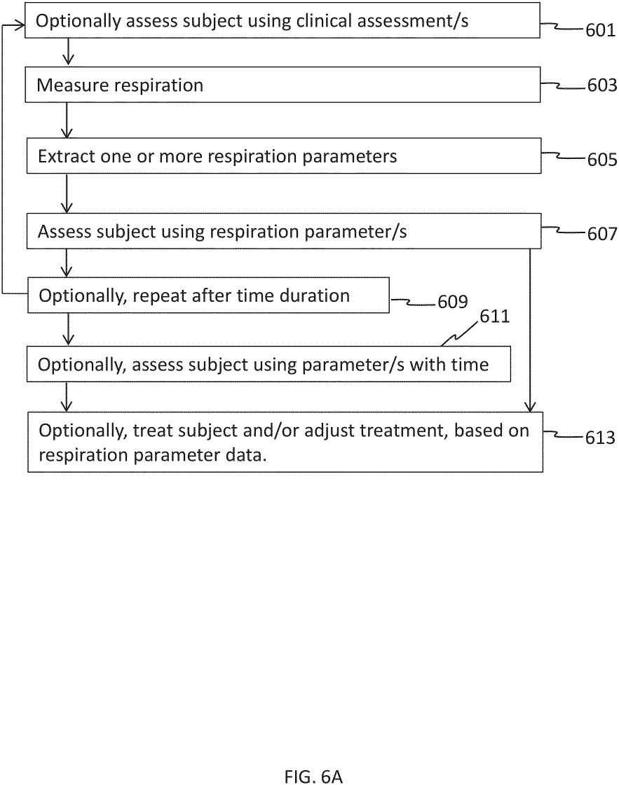

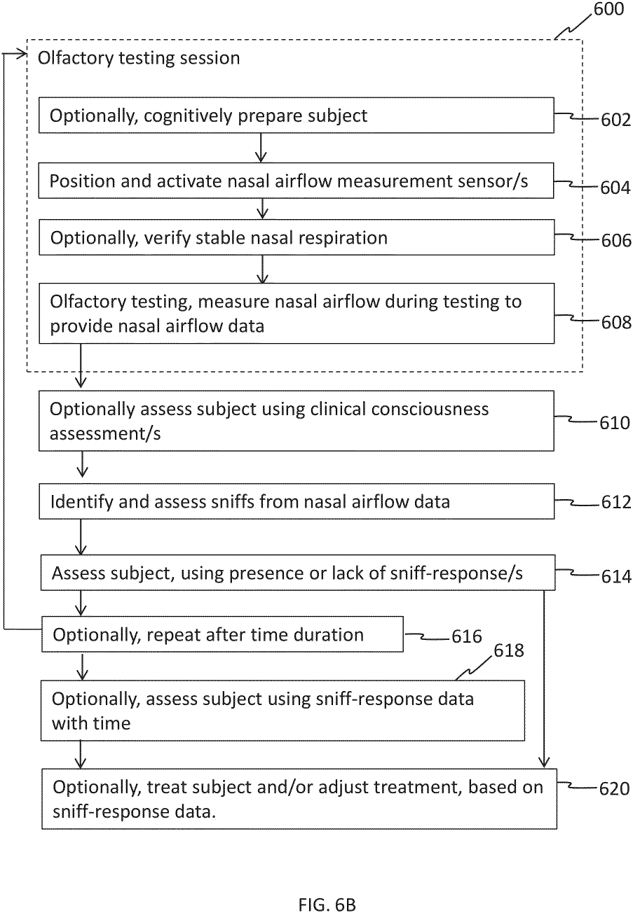

At 100, in some embodiments, olfactory testing is performed on a subject. Where the olfactory testing includes one or more feature as illustrated in and/or described regarding FIG. 4 and/or one or more of steps 600-608 FIG. 6B.

At 102, in some embodiments, during olfactory testing, subject nasal airflow is measured e.g. by one or more sensor (e.g. sensor/s 204FIG. 2, sensor/s 304FIG. 3).

At 104, in some embodiments, the subject is assessed based on nasal airflow measurements.

For example, where assessment includes determining a likelihood that the subject is in one or more particular state of consciousness and/or a likelihood of recover and/or survival of the subject.

Referring to FIG. 1A, in some embodiments, sniff data for individual subjects is assessed by comparison to group averages for the group in which the individual is categorized (at least initially).

Alternately, in some embodiments, sniff data for an individual subject is assessed using a threshold derived from averages from a group with a higher level of consciousness. Potentially, such a threshold indicates that the individual is on a threshold between groups and/or soon to transfer from a group to a higher level of consciousness and/or is misdiagnosed in a lower level of consciousness group than is correct. For example, in some embodiments, VS/UWS and/or MCS subjects are assessed using a sniff response volume determined from healthy conscious subjects.

### Exemplary Systems for Subject Assessment

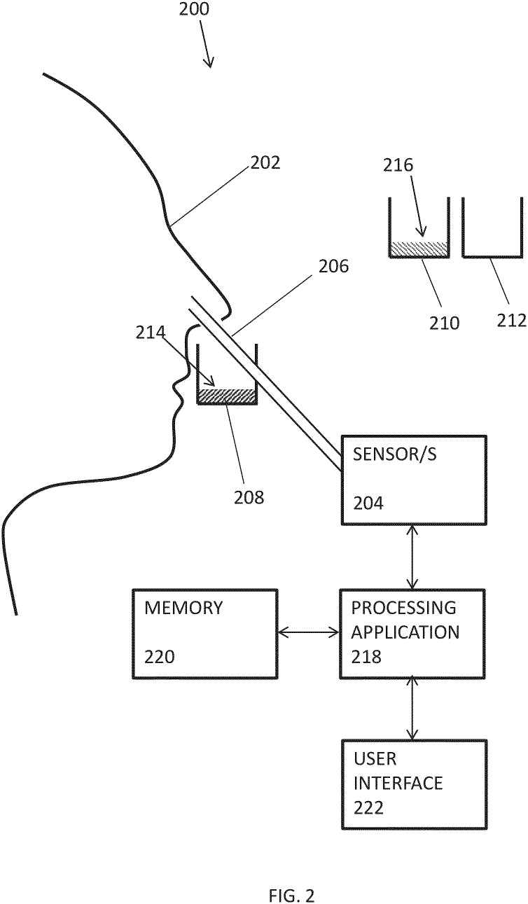

FIG. 2 is a system 200 for assessing a subject, according to some embodiments of the invention.

In some embodiments, system 200 includes one or more sensor 204, for measurement of respiration e.g. of nasal airflow of the subject 202. In some embodiments, sensor/s 204 include a spirometer. In some embodiments, airflow sensor/s 204 are fluidly connected to a cannula 206 which is placed within the subject’s nasal passageway.

Alternatively or additionally, in some embodiments, airflow is measured by measuring movement of the abdomen during respiration, exemplary sensors being piezoelectric elements responding to changes in length associated with respiration of the subject.

Alternatively or additionally, in some embodiments, temperature of nasal airflow is measured, for example by thermistor/s placed within the nasal air flow (and e.g. not in contact with the skin).

Alternatively or additionally, in some embodiments, a pneumotachometer is used to measure differential pressure of the nasal air flow. Where, in some embodiments, the differential pressure is converted into a voltage signal using a spirometer.

In an exemplary embodiment, nasal airflow is measured using a nasal cannula (e.g. 1103, Teleflex medical) linked directly to a spirometer (e.g. ML141, ADInstruments, H₂O resolution = 15.6 µV). Where the spirometer, in some embodiments, converts airflow into a voltage signal. In an exemplary embodiment, the airflow voltage signal is amplified by an instrumentation amplifier (e.g. PowerLab 16SP Monitoring System, ADInstruments).

In some embodiments, data is collected by sampling the airflow voltage signal. In some embodiments, the airflow signal is sampled at 100-10,000 Hz, or 500-200 Hz, or at about 1000 Hz, or lower or higher or intermediate ranges or sampling rates. In an exemplary embodiment, sampling is at 1000 Hz.

In an exemplary embodiment, sampling is using LabChart software (ADInstruments).

Optionally, in some embodiments, system 200 includes one or more container 208, 210, 212 used for olfactory testing. In come embodiments, one or more containers holds one or more scent source 214, 216, and/or system containers include one or more blank container 212. In some embodiments, one or more container is a solid scent source.

In an exemplary embodiment containers are sniff-jars. In an exemplary embodiments, odorants are soaked onto a cotton pad placed in a container (e.g. sniff-jar) and, in some embodiments, a blank container is a container with a cotton pad alone.

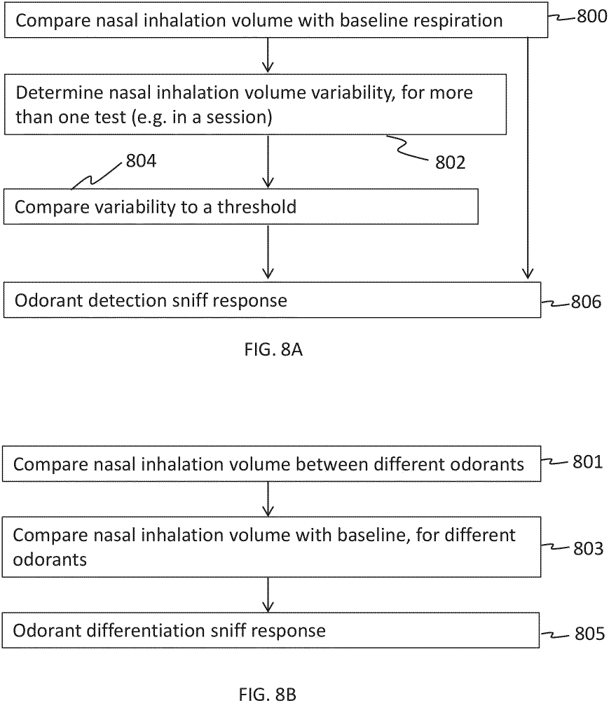

In some embodiments, system 200 includes a processing application 218 which, in some embodiments, receives sensor data from sensor/s 204. In some embodiments, processing application 218 performs one or more method step of methods described in this document e.g. one or more step of the method of one or more of FIG. 1B, FIG. 1C, FIG. 6A, FIG. 6B, FIG. 8A-14.

In some embodiments, system 200 includes one or more memory 220, where, for example, sensor data is stored and/or other data e.g. patient data e.g. previous measurement/s and/or assessments of the patient. In some embodiments, memory holds one or more threshold.

Optionally, in some embodiments, system 200 includes one or more user interface 222. In some embodiments, user interface 222 provides feedback to a user regarding sensor measurements. In some embodiments, user interface 222 prompts to user as to which olfactory test to perform and/or when to present an olfactory test.

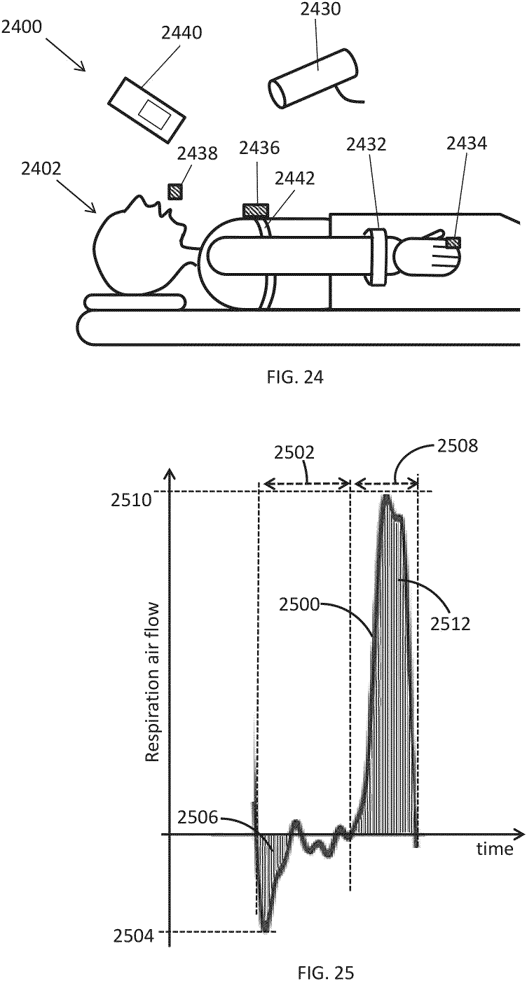

FIG. 24 is a system 2400 for assessing a subject, according to some embodiments of the invention.

In some embodiments, system 2400 includes one or more sensor 2404, 2430, 2432, 2434, 2436, 2438 for measurement of a subject 2402.

In some embodiments, respiration of subject 2402 is measured using one or more sensor. For example, in some embodiments, sensor 2438 measures subject respiration air flow. Where, in an exemplary embodiment, sensor 2438 is a spirometer which, in some embodiments is attached to a nasal cannula for measurement of nasal air flow.

Alternatively or additionally to nasal air flow, in some embodiments, oral air flow is measured.

In some embodiments, respiration is measured using one or more movement sensor 2436 and/or pressure sensor 2436 and/or one or more optical sensor 2430.

For example, in some embodiments, a pressure sensor 2436 senses changes in pressure on the sensor associated with respiration. In some embodiments, pressure sensor 2436 is held in contact with and/or in position on a torso of subject 2402 e.g. by a strap 2442. Alternatively or additionally, in some embodiments, a sensor 2436 e.g. coupled to the subject’s torso is a movement sensor sensing movement of the torso e.g. including movement/s associated with respiration.

In some embodiments, system 2400 includes one or more additional sensor, e.g. for physiological measurement of subject 2402. For example, one or more of a blood oxygenation sensor (e.g. located on a subject’s finger 2434), a temperature sensor, a cardiac cycle sensor.

In some embodiments, optical sensor 2430 detects and/or measures respiration and/or other subject parameters (e.g. other movements of the subject).

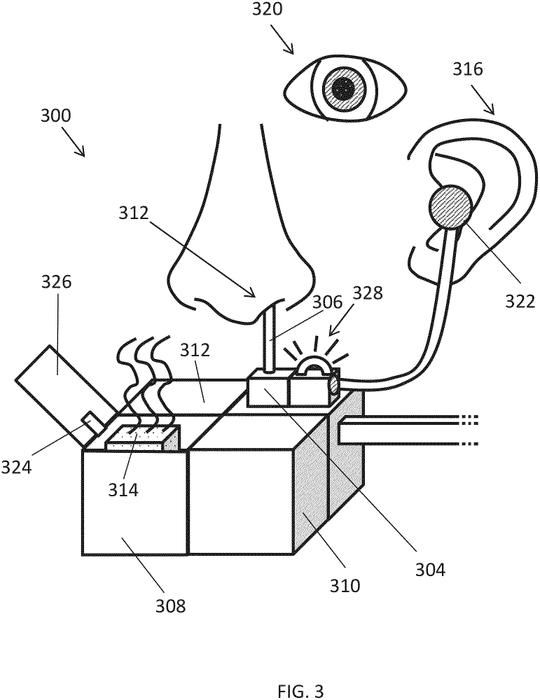

FIG. 3 is a system 300 for assessing a subject, according to some embodiments of the invention.

In some embodiments, system includes one or more sensor 304 to measure nasal air flow. In some embodiments, sensor 304 is connected to a cannula 306 which is, in some embodiments, positioned within a subject’s nostril 312. Where sensor 304, in some embodiments, includes one or more feature as illustrated and/or described regarding sensor/s 204FIG. 2. Where cannula 304, in some embodiments includes one or more feature as illustrated and/or described regarding cannula 204FIG. 2.

In some embodiments, system 300 includes one or more container 308, 310, 312. In some embodiments, one or more of container/s 308, 310, 312, hold one or more scent source 314.

In some embodiments, one or more part of olfactory testing of a subject is performed automatically. For example, in some embodiments, presentation of an olfactory test to the subject is automatic.

For example, in some embodiments, system 300 includes one or more actuator 324 configured to presents an olfactory test to the subject. For example, in some embodiments, actuator/s open a container containing a scent sample, e.g. as illustrated by actuator 324, open container 308, and scent source 314 where actuator/s 324 have opened a cover 326 of container 308 to expose the subject’s nose 312 to scent emitted by scent source 314.

Alternatively or additionally to opening a container, in some embodiments, actuator/s move a container (and/or scent source e.g. solid scent source) in space towards the subject e.g. towards the subject’s nose 312.

In some embodiments, actuator/s move a scent itself (e.g. blowing scented air) towards the subject. For example, in some embodiments, system 300 includes an olfactometer.

In some embodiments, system 300 includes one or more interface, for interacting with the subject. For example, to cognitively prepare subject (e.g. step 602FIG. 6B) and/or to stimulate the subject (e.g. as described regarding step 620FIG. 6B).

For example, in some embodiments, system 300 includes one or more speaker 322 e.g. to provide aural cue/s to the subject 316. For example, in some embodiments, system 300 includes one or more light source and/or screen 328, e.g. to provide visual cue/s to the subject 320. Additionally or alternatively, in some embodiments, one or more of the interfaces 322, 328 provide cue/s to a user e.g. in setting up the system and/or operating the system.

### Exemplary Olfactory Testing

FIG. 4 is a simplified schematic illustration of an olfactory testing session, according to some embodiments of the invention.

In some embodiments, an olfactory testing session includes at least one presentation. In an exemplary embodiment, a testing session includes a plurality of presentations, for example, 2-100, or 5-50, or 10-40, or about 30 presentations or lower or higher or intermediate numbers of presentations or ranges.

In some embodiments, a single type of presentation is performed, for example, a single odorant is presented.

In some embodiments, a presentation is for a time duration. Where, in some embodiments, the time duration is for about 5 seconds, or 1-30 seconds, or 1-10 seconds, or lower, or higher, or intermediate ranges, or time durations.

In some embodiments, each presentation is followed by a resting time where no presentation is performed. In some embodiments the resting time is about 30 seconds, or 10 seconds - 2 minutes, or 10 seconds to 1 minute, or lower, or higher, or intermediate ranges, or time durations.

In some embodiments, more than one type of presentation is performed, for example, where different odors are presented e.g. two different odors e.g. a pleasant odor and an unpleasant odor. In some embodiments, different types of pleasant and/or unpleasant odor are presented e.g. different odor, in some embodiments, termed a different type of presentation.

In some embodiments, cognitive presentations are performed, for example, were a subject is cognitively prepared for olfactory testing, for example, using a method of communication which is not olfactory (e.g. prepared aurally and/or visually and/or haptically) and then a blank presentation is performed.

In an exemplary embodiment, three types of presentation are performed; a pleasant odorant, a unpleasant odorants and blank presentations. In an exemplary embodiment, different types of presentation are performed randomly, each type of presentation being performed multiple times.

In an exemplary embodiment, each odorant is presented to the subject about 10 times, as long as nasal respiration is evident.

In some embodiments, a presentation includes performing an action which is able to be sensed by the average person. In some embodiments, the action exposes the subject to a scent.

In an exemplary embodiment, a presentation is performed by bringing a container into the subject’s field of view and then, in some embodiments, placing the container under the nose of the subject (e.g. without touching the subject). In some embodiments, the container contains a scent source.

Alternatively, in some embodiments, the action includes a haptic cue to the subject, e.g. touching the subject e.g. with a container, e.g. blowing air (optionally containing scent) towards the subject.

In some embodiments, timing of a presentation is based on respiration of the subject. For example, in some embodiments, presentation/s are synchronized with respiration of the subject. In an exemplary embodiment, presentations are performed at an end of an exhale so that the subject is exposed to the odorant in the following inhale. For example, at a final 1%-50% of an exhale and/or at a final 50 ms-1 second of an exhale.

Exemplary odorants include commercial odorant mixtures e.g. pleasant “shampoo” and unpleasant “rotten fish” e.g. as provided by Sensale, Ramat Gan, Israel. Exemplary odorants include pure odorant molecules e.g. the pleasant PhenylEthyl Alcohol (PEA), CAS #102-20-5, that smells like rose, and the slightly unpleasant decanoic acid, CAS #334-48-5, that smells like crayons, both from Sigma-Aldrich, Rehovot, Israel.

FIG. 5A is a simplified schematic of nasal air flow with time before and during an olfactory test, according to some embodiments of the invention.

In some embodiments, FIG. 5A shows, with time, a trace of nasal respiration e.g. recorded using a nasal cannula connected to a spirometer and amplifier.

In FIGS. 5A, 3 baseline breaths precede an olfactory presentation 500 e.g. of an unpleasant odorant. In some embodiments, the dashed line denotes odorant onset and bar 502 represents odorant duration. Sniff 1, Sniff 2, and Sniff 3 annotations illustrate a inhalations following the olfactory presentation (sniffing).

For example, as illustrated in FIG. 5A, in some embodiments, a subject’s sniff response continues in time after cessation of a presentation.

FIG. 5B is a simplified schematic of nasal air flow with time for a MCS subject, according to some embodiments of the invention.

FIG. 5C is a simplified schematic of nasal air flow with time for a VS/UWS subject, according to some embodiments of the invention.

### Exemplary Detailed Method of Subject Assessment

FIG. 6A is a method of assessing a subject, according to some embodiments of the invention.

At 601, in some embodiments, the subject is assessed using clinical assessment/s. For example, one or more medical assessment to identify and/or diagnose medical issue/s. For example, one or more clinical consciousness assessment e.g. including one or more feature as illustrated in and/or described regarding step 610FIG. 6B.

At 603, in some embodiments, respiration of the subject is measured.

In some embodiments, respiration is measured by measuring nasal airflow, for example, according to one or more feature illustrated in and/or described regarding step 604FIG. 6B.

Alternatively or additionally to measurement of nasal airflow, in some embodiments, respiration is measured by measuring oral airflow.

Alternatively or additionally, in some embodiments, respiration is measured using one or more other measurement method. For example, by one or more sensor as illustrated in and/or described regarding sensors 2430, 2432, 2434, 2436, 2438, 2440FIG. 24. For example, using one or more of optical sensor/s (e.g. to measure breathing motion), movement sensor/s (e.g. to measure respiration movement of the subject’s body), and pressure sensor/s.

Optionally, in some embodiments, airflow measurements are used to provide respiration parameter/s and/or are collected after checking that the subject has stable respiration. For example, as described regarding step 1002FIG. 10.

In some embodiments, measurement using one or more measurement technique is for a time period e.g. in some embodiments measurements are collected sporadically and/or periodically. In some embodiments, measurement using one or more measurement technique is continuous.

At 605, in some embodiments, one or more respiration parameter is extracted from airflow measurement data. Where, in some embodiments, (e.g. as described elsewhere in this document) respiration parameter/s include average and/or variability of respiration feature/s (respiration feature/s as illustrated in and/or described regarding FIG. 25.

In some embodiments, respiration measurement/s other than respiration airflow measurements have a waveform similar to that of airflow measurements e.g. as illustrated in FIG. 25. Where, in some embodiments, respiration feature/s for non-airflow measurements are extracted as described regarding and/or illustrated in FIG. 25.

Optionally, in some embodiments, to extract respiration feature/s and/or parameter/s (e.g. sniff response) from measurement data, noise is reduced and/or removed from sensor signal/s, for example, as described regarding step 902FIG. 9.

Optionally, in some embodiments, to extract respiration feature/s and/or parameter/s (e.g. sniff response) from measurement data (and/or from measurement data from which noise has been reduced and/or removed) respirations are identified, for example, as described regarding step 904FIG. 9.

At 607, in some embodiments, the subject is assessed based on respiration parameter/s extracted from measurements collected during a time period and/or an immediately preceding time the assessment. For example, in the case where measurement is periodic (e.g. as described regarding step 609), the assessment at 607 is based on the previous measurement session.

In some embodiments, for example, where measurements are collected continuously, assessment is made based on measurement parameter/s extracted from measurements at different time periods. For example, in some embodiments, at step 607 assessment is made for a different time period than that of the assessment at step 611.

At 609, optionally, in some embodiments, one or more of steps 601-607 are repeated.

For example, in some embodiments, respiration measurements are collected periodically. For example, at least once a day, or 1-5 times a day, or 1-3 times a day or once every other day, or at least twice a week, or 1-20 times a week, or lower or higher or intermediate frequencies or ranges.

For example, in some embodiments, measurements are collected continuously. For example, where respiration measurements used to determine respiration parameters and/or to assess the subject are provided by patient monitoring equipment. For example, respiration monitor data, ECG data.

At 611, optionally, in some embodiments, the subject is assessed.

In some embodiments, the subject is assessed using a collection of assessments over time. A potential advantage being reduced likelihood of overlooking the possible interdependence of measures obtained from the same individual, including, in some embodiments, for example, if the subject transfers to a different level of consciousness.

In some embodiments, the subject is assessed using respiration parameters determined from different time periods. For example, from different testing sessions (e.g. in the case of periodic measurement of respiration). For example, where time periods, for one or more respiration parameter are non-overlapping. For example, where, for one or more respiration parameter, the time periods are overlapping.

In some embodiments, the subject is assessed using an average of different assessments.

In some embodiments, the subject is assessed using a strongest session in a time period. For example, in some embodiments, over a time period of 1 day - 1 year, or 1 day - 6 months, or 1 week to 6 months, or 1 week to 3 months, or lower or higher or intermediate times or ranges, a strongest measured sniff response is used to assess the subject.

At 613, optionally, in some embodiments, the subject is treated and/or treatment of the subject is changed, based on the assessment performed at step 607 and/or at step 611.

In some embodiments, treatment is adjusted and/or based on assessment. For example, pain management (e.g. pain management medication). For example, end-of-life decisions.

FIG. 6B is a method of assessing a subject, according to some embodiments of the invention.

At 600, in some embodiments, an olfactory testing session, is performed on a subject, where, for example, the testing includes one or more feature as illustrated in and/or described regarding FIG. 4. In some embodiments, olfactory testing includes one or more of the steps 602, 604, 606 and 608.

At 602, optionally, in some embodiments, the subject is cognitively prepared for olfactory testing. For example, in some embodiments, it is explained to the subject that odorants will be presented (e.g. using sniff-jars) and that nasal respiration will be monitored during the session. In some embodiments, this is repeated regardless of indication/s from the subject that the subject heard and/or understood what is said.

In some embodiments, cognitive preparation is performed only where cognitive olfactory testing is performed.

At 604, in some embodiments, one or more nasal airflow measurement sensor is positioned and/or activated. For example, in some embodiments, a nasal cannula connected to a sensor is applied to the subject’s nostrils in order to record the subject’s nasal respiration. In some embodiments, subject nasal airflow during olfactory testing is measured (e.g. where measuring includes one or more feature as illustrated in and/or described regarding sensor/s 204FIG. 2 and/or sensor/s 304FIG. 3) to provide nasal airflow data.

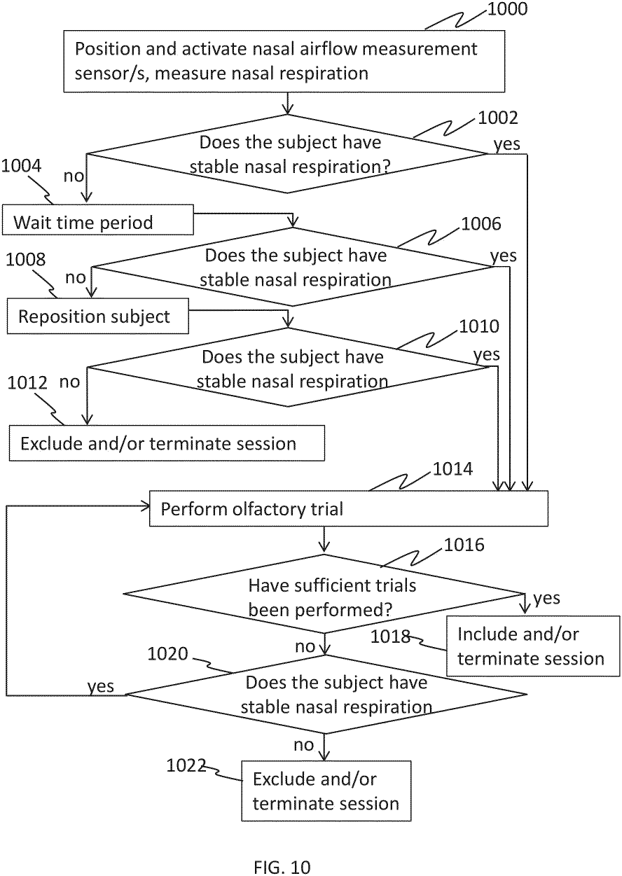

At 606, in some embodiments, stable nasal respiration is then verified, e.g. including one or more feature as illustrated in and/or described regarding one or more of steps 1002-1012 FIG. 10. In some embodiments, if stable nasal respiration is lacking, olfactory testing is terminated. In some embodiments, step 606 is performed before step 604, stable nasal respiration is verified before positioning and/or activating nasal airflow measurement sensor/s.

At 608, in some embodiments, an olfactory testing session is performed, where, in some embodiments, nasal airflow is measured during the testing. Where olfactory testing, in some embodiments, includes one or more feature as illustrated in and/or described regarding FIG. 4.

At 610, in some embodiments, the subject is assessed using clinical consciousness assessment/s.

For example, to determine between vegetative state/unresponsive wakefulness syndrome (VS/UWS) reflecting no signs of consciousness, or minimally conscious state (MCS) reflecting inconsistent but reproducible evidence of consciousness.

In an exemplary embodiment, assessment is directly following an olfactory testing session. Alternatively or additionally, in some embodiments, the subject is evaluated e.g. periodically using one or more clinical consciousness assessment.

In some embodiments, assessment of the subject is using the Coma Recovery Scale Revised (CRS-R) (e.g. as described in Background art reference 19).

The CRS-R evaluates the presence or absence of responses to auditory, visual, motor, oromotor, communication and arousal function. CRS-R is quantitative with scores ranging from 0 (lowest level of consciousness) to 23 (highest level of consciousness), and also qualitative with 4 levels: coma, VS/UWS, MCS and emergence from MCS, with specific behaviors defining each level. The CNC evaluates the occurrence of responses to visual, auditory, command following, threat response, olfactory, tactile, pain, vocalization.

Alternatively or additionally, in some embodiments, the subject is assessed using the Coma-Near Coma scale (CNC)²⁰_{.}

The CNC is quantitative with scores ranging from 4 (lowest level of consciousness) to 0 (highest level of consciousness), and also qualitative with 5 levels: extreme coma (3.5-4), marked coma (2.9-3.49), moderate coma (2.01-2.89), near coma (0.9-2), no coma (0-0.89).

In some embodiments, CNC qualitative levels are converted, based on Rappaport et al.,²⁰ as follows: extreme coma and marked coma = VS/UWS; moderate coma and near coma = MCS; No coma = emergence from MCS.

Alternatively or additionally, in some embodiments, the subject is assessed using the Loewenstein Communication Scale (LCS)⁵¹.

LCS evaluates five hierarchical functions: mobility, respiration, visual responsiveness, auditory comprehension and linguistic skills (verbal or alternative). The LCS is quantitative with scores ranging from 0 to 100, where scores up to 20 are considered VS/UWS and scores above 20 are considered MCS.

In an exemplary embodiment, CRS-R and/or CNC are used directly after olfactory testing sessions and LCS is used periodically.

In some embodiments, for each testing session, the subject is assessed using clinical consciousness assessment/s e.g. to take into account the possibility of a subject having a fluctuating consciousness level.

Optionally in some embodiments, the subject is assessed to identify and/or assess brain structure related injury and/or lack of function. For example, olfaction-related brain structure injury.

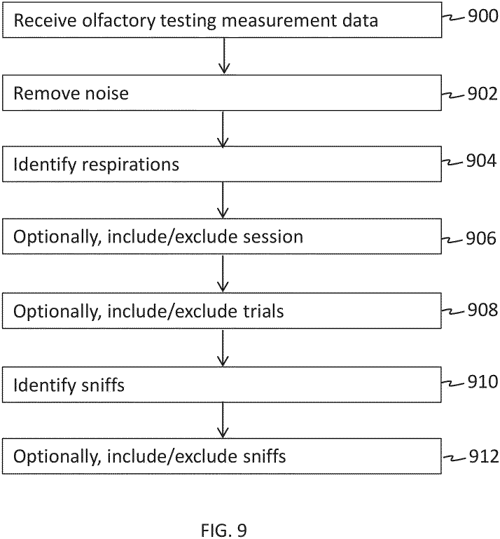

At 612, in some embodiments, presence or absence of sniff response/s are identified. For example, where presence and/or absence is determined according to one or more feature as illustrated in and/or described regarding FIG. 9.

At 614, in some embodiments, the subject is assessed, using presence and/or lack of sniff response/s. Optionally, in some embodiments, clinical consciousness assessments are used in the assessment.

In some embodiments, the subject is assessed “by session” to take into account the possibility of a subject having a fluctuating consciousness level.

Optionally, in some embodiments, the subject is assessed using baseline respiration measurements e.g. in absence of olfactory stimulation. For example, in some embodiments, one or more respiration parameter is determined and used to assess the subject e.g. alternatively or additionally to using sniff response assessment. For example, in some embodiment (e.g. referring to FIG. 1A) it is assumed that the lower level of consciousness and/or functionality a subject has, the lower baseline respiration variability and/or lower baseline respiration volume of the subject. For example, referring to FIG. 5B and FIG. 5C, the VS/UWS subject has lower respiration variability and lower baseline respiration volume than the MCS subject.

At 616, optionally, in some embodiments, after a time duration, assessment of the subject (e.g. by performing steps 602-612 again) is repeated, one or more times.

Where, in some embodiments, the time duration is 1 day - 1 month, or 1 day to 1 year, or 1 day - 6 months, or 1 week to 6 months, or 1 week to 3 months, or lower or higher or intermediate times or ranges.

Where, in some embodiments, the assessment is repeated a plurality of times, for example, until recovery of the subject. For example, if the time duration is 1 month, in some embodiments, the assessment of the subject is repeated once a month for a plurality of months.

At 618, optionally, in some embodiments, the subject is assessed using repeated assessments (e.g. as described in step 616) and/or using sniff response data over time.

In some embodiments, the subject is assessed using a collection of assessments over time. Potentially preventing overlooking the possible interdependence of measures obtained from the same individual, including, in some embodiments, for example, if the subject transfers to a different level of consciousness.

In some embodiments, the subject is assessed using an average of different assessments.

In some embodiments, the subject is assessed using a strongest session in a time period. For example, in some embodiments, over a time period of 1 day - 1 year, or 1 day - 6 months, or 1 week to 6 months, or 1 week to 3 months, or lower or higher or intermediate times or ranges, a strongest measured sniff response is used to assess the subject.

At 620, in some embodiments, the subject is treated and/or treatment of the subject is changed, based on of subject sniff response assessment (e.g. of step 614 and/or step 618).

In some embodiments, the subject is stimulated and effectiveness of the stimulation is assessed. In some embodiments, stimulation is changed based on effectiveness, as assessed e.g. in steps 614 and/or 618. Where, in some embodiments stimulation includes one or more of aural, visual, haptic and olfactory stimulation. In some embodiments, stimulation is automatic e.g. including one or more feature as described regarding interfaces 322, 328, FIG. 3.

In some embodiments, treatment is adjusted and/or based on assessment. For example, pain management (e.g. pain management medication). For example, end-of-life decisions.

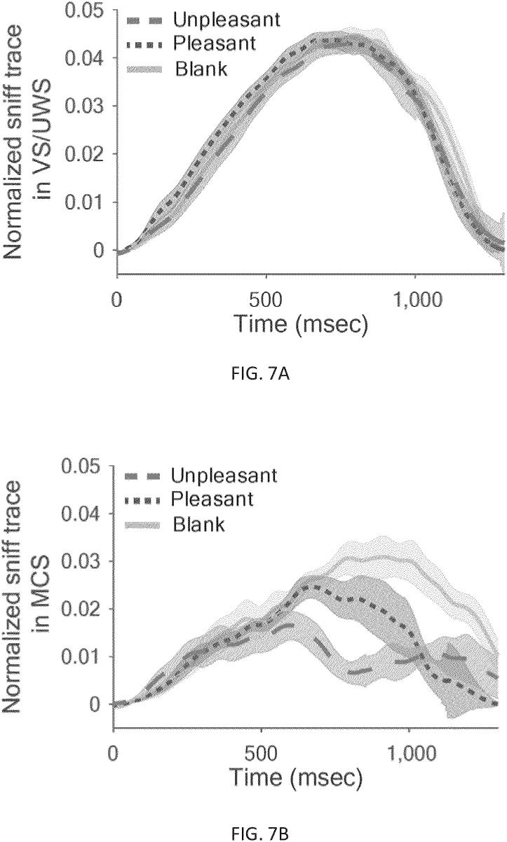

FIG. 7A is a simplified schematic of a normalized sniff trace for olfactory tests, for a vegetative state/unresponsive wakefulness syndrome (VS/UWS) subject, according to some embodiments of the invention.

FIG. 7A, in some embodiments, illustrates exemplary data from a subject lacking a sniff response.

Where, FIG. 7A shows results of a session where:
A pleasant odorant was presented 11 times and the normalized sniff volume, for the pleasant odorant = 1.00 ± 0.08;
An unpleasant odorant was presented 12 times and the normalized sniff volume for the unpleasant odorant = 0.94 ± 0.1; and
A blank presentation was performed 11 times and the normalized sniff volume, for the blank presentation = 1.00 ± 0.09.

FIG. 7B is a simplified schematic of a normalized sniff trace for olfactory tests, for a minimally conscious state (MCS) subject, according to some embodiments of the invention.

FIG. 7B, in some embodiments, illustrates exemplary data from a subject with an intact sniff response.

Where, FIG. 7B shows results of a session where:
A pleasant odorant was presented 7 times and the normalized sniff volume, for the pleasant odorant = 0.68 ± 0.22;
An unpleasant odorant was presented 7 times and the normalized sniff volume for the unpleasant odorant = 0.56 ± 0.19; and
A blank presentation was performed 6 times and the normalized sniff volume, for the blank presentation = 0.84 ± 0.21.

Visually, it appears, for the two exemplary subjects, that the VS/UWS patient (FIG. 7A) has lower sniff volume difference from baseline and variability of the sniff volume is larger than the MCS subject (FIG. 7B).

### Exemplary Sniff Response Definitions

In some embodiments, more than one type of sniff response is defined. Where, in some embodiments, exemplary sniff responses include one or more of an odorant detection sniff response, a cognitively-driven sniff response and an odorant differentiation sniff response.

Where, in some embodiments, an odorant detection sniff response is where nasal airflow changes in response to presence of an odorant.

Where, in some embodiments, an odorant differentiation sniff response is where nasal airflow has a differential response in response to different odorants. For example, reduced nasal airflow for unpleasant versus pleasant odorants.

FIG. 8A is a method of evaluating an odorant detection sniff response and/or a cognitively-driven sniff response of a subject, according to some embodiments of the invention.

At 800, in some embodiments, for each trial, nasal inhalation volume/s after olfactory test presentation (also herein termed “sniff/s” or “sniff volume/s”) are identified.

At 805, nasal inhalation volume (and/or nasal exhalation volume) is compared to baseline respiration. For example, in some embodiments, a ratio between sniff volume and baseline respiration (also herein termed normalized sniff volume) is compared to a threshold.

In some embodiments, the threshold is based on measurements collected from healthy conscious subjects. For example, in an exemplary embodiment, using previously collected measurements as described in background art number 17 (incorporated by reference in its entirety).

In some embodiments, the threshold for change in sniff volume was determined using measurements of pleasant (PhenylEthyl Alcohol) and unpleasant (Valeric acid) odorants in relation to clean air in healthy conscious subjects. For example, sniff volume (integral) values measured using nasal cannula measurement.

In some embodiments, a threshold determined using pure odorant molecules is used for determining sniff response in olfactory testing using odorant mixes.

In some embodiments, if sniff measurements for subjects are measured using a different measurement method, a threshold is determined using that measurement method in conscious subjects. In some embodiments, for assessment of subjects not expected to have odorant differentiation (e.g. DoC subjects) an average of measurements of pleasant and unpleasant odorants in conscious subjects is used to determine the threshold e.g. using the below equation: Sniff volume threshold = [(Vₚₗₑₐₛₐₙₜ + Vᵤₙₚₗₑₐₛₐₙt)/2]/V_{clean_air}

Where Vₚₗₑₐₛₐₙₜ is average sniff volume for pleasant odorants, Vᵤₙₚₗₑₐₛₐₙₜ is average sniff volume for unpleasant odorants and V_{clean_air} is average nasal inhalation volume for clear air.

Measurement data from background art number 17 gives Vₚₗₑₐₛₐₙₜ = 0.837, Vᵤₙₚₗₑₐₛₐₙₜ= 0.641, V_{clean}_ₐᵢᵣ= 0.86 and

Exemplary sniff volume threshold = [(0.837 + 0.641)/2]/0.86 = 0.8593 which is ∼15% decrease.

In an exemplary embodiment, the threshold is 15% where, for example, if the normalized sniff volume is at least 15% less than averaged baseline respiration of the subject, it is determined that, at 810, the subject has an odorant detection sniff response.

In some embodiments, the threshold is 1-50%, or 10-20%, or 12-18%, or about 15%, or lower or higher or intermediate percentages or ranges.

In some embodiments exhalation measurements are used, alternatively or additionally to using inhalation measurements. For example, where, in some embodiments, if the normalized exhalation volume after odor presentation is at least a threshold percentage less than averaged baseline respiration of the subject (e.g. calculated using exhalation measurements of baseline) it is determined that, at 810, the subject has an odorant detection sniff response. Where, in some embodiments, the threshold percentage is 1-50%, or 10-20%, or 12-18%, or about 15% or lower or higher or intermediate percentages or ranges.

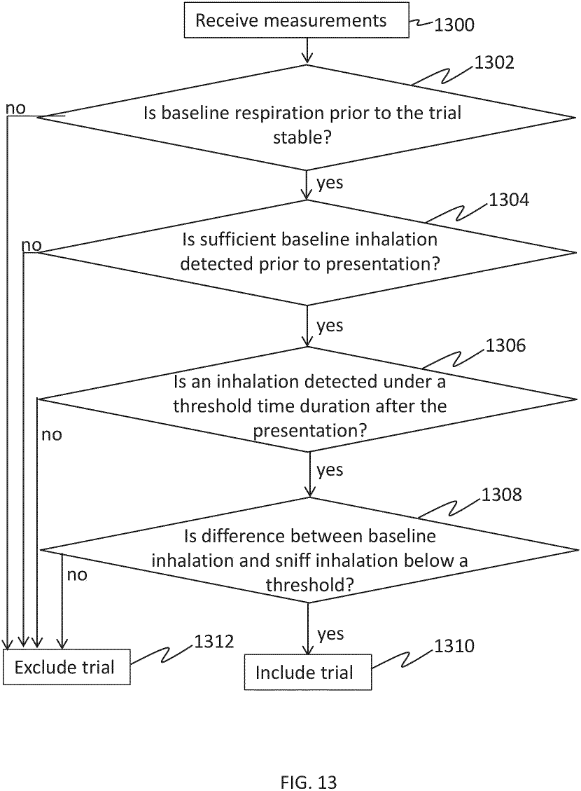

At 806, in some embodiments, variability of sniff volume for more than one trial in a session is determined e.g. for all included trials in a session (trial inclusion criteria e.g. as described in FIG. 13).

At 808, in some embodiments, the variability of nasal inhalation is compared with a sniff variability threshold. In some embodiments, if the variability is larger than the threshold, at 810, it is determined that the subject has a sniff response.

In some embodiments, the threshold is based on measurements from DoC subject/s. In an exemplary embodiment, the sniff variability threshold for DoC subjects is based on MCS subject variability measurements. In an exemplary embodiment, the sniff variability threshold is a multiple (e.g. 1.5-5, or 1.4-3 or lower or higher or intermediate ranges or multiples) of an averaged standard deviation of sniff volume for MCS subjects. In an exemplary embodiment, the sniff variability threshold is double an averaged standard deviation of sniff volumes for all MCS subjects (including those which did not have a sniff response) which was 0.35.

In some embodiments, the sniff variability threshold for odorant detection is 0.1-1, or 0.1-0.5, or 0.2-0.4, or 0.3-0.4 standard deviation across trials, or lower or higher or intermediate standard deviations or ranges.

In an exemplary embodiment, the sniff variability threshold for odorant detection is that the nasal inhalation volume standard deviation (SD) across all trials in the session (e.g. all included trials in the session) is larger than 0.35.

In some embodiments, threshold/s for cognitively-driven sniff response are the same as threshold/s for odorant detection sniff response.

In some embodiments, a subject is determined to have an odorant detection and/or cognitively-driven sniff response if one of sniff volume (e.g. as described in step 805) and sniff variability (e.g. as described in step 808).

In some embodiments, a subject is determined to have an odorant detection and/or cognitively-driven sniff response only when both sniff volume (e.g. as described in step 805) and sniff variability (e.g. as described in step 808) threshold comparisons are satisfied.

FIG. 8B is a is a method of evaluating an odorant differentiation sniff response of a subject, according to some embodiments of the invention.

At 801, in some embodiments, nasal inhalation volume between odorants is compared. For example, different odorant types (e.g. pleasant and unpleasant).

At 803, in some embodiments, nasal inhalation volume, normalized with baseline (e.g. average baseline inhalation volume) is compared.

At 804, in some embodiments, the subject is determined to have an odorant differentiation sniff response if a difference in magnitude (e.g. normalized magnitude) of sniffs for different odorants is above a threshold. Where in some embodiments, the odorant differentiation is threshold is 5-50%, or 5-30%, or 15-25% difference in magnitude, or lower or higher or intermediate ranges or percentages. In an exemplary embodiment, the odorant differentiation threshold is 20% or about 20%.

Optionally, additionally, in some embodiments, the subject is determined to have an odorant differentiation sniff response if sniff volume, for example, with respect to baseline, (e.g. normalized sniff volume) for both odorants is below a threshold. For example, where both odorants are lower in volume than baseline (normalized volumes are both <1).

### Exemplary Processing of Olfactory Testing Data

FIG. 9 is a method of processing olfactory testing data, according to some embodiments of the invention.

At 900, in some embodiments, olfactory testing measurement data is received. In some embodiments, the data includes nasal respiration data. In some embodiments, the data includes timing data of olfactory tests.

At 902, in some embodiments, in some embodiments, nasal airflow data is filtered e.g. to remove noise, for example, low pass filtered e.g. to remove high frequency noise.

In some embodiments, an equiripple low pass filter (LPF) is applied. In some embodiments, filtering is to remove high frequency noise of above 20 Hz, or above 25 Hz, or above 30 Hz. For example, in some embodiments, a LPF with pass frequencies below 10 Hz and a stop Frequency of 20 Hz is used.

In an exemplary embodiment, the nasal airflow data is filtered with an equiripple filter with pass frequencies below 10 Hz, a stop Frequency of 20 Hz and allowed ripple amplitude of 1 dB where the stop amplitude attenuation is 60 dB and the filter number of coefficients is 224.

At 904, in some embodiments, respiration (inhales and exhales) are identified from the nasal airflow data.

For example, in some embodiments, the nasal airflow data is low pass filtered, e.g. to provide an inhale/exhale waveform. In some embodiments, filtering is to remove portion/s of the signal above 5 Hz, or above 10 Hz. For example, in some embodiments, a LPF with pass frequencies below 5 Hz and a stop Frequency of 6 Hz is used.

In an exemplary embodiment, the nasal airflow data is filtered with an equiripple filter with pass frequencies below 5 Hz, a stop Frequency of 6 Hz and allowed ripple amplitude of 1 dB where the stop amplitude attenuation is 60 dB and the filter number of coefficients is 1975.

In some embodiments, hysteresis is applied (e.g. after filtering) to identify inhales and/or exhales. Where, in an exemplary embodiment, inhales and/or exhales were identified using the smallest of the two of: a 5 mV threshold and a threshold of 5% of max-min values. In some embodiments, hysteresis to identify inhales and/or exhales was applied with a minimum duration time window of 250 msec.

In alternative embodiments, one of the two of a Voltage threshold and a percentage threshold are used.

In some embodiments, the voltage threshold is 1-10 mV, or 2-7 mV or higher or lower or intermediate voltages or ranges.

In some embodiments, the percentage threshold is 1-20%, or 1-10%, or 2-7%, or lower or higher or intermediate percentages or ranges.

In some embodiments, the minimum duration time window is 50-300 msec or 250-500 msec, or lower or higher or intermediate times or ranges.

Alternative and/or additional methods of inhale and/or exhale identification include:
● Detection by eye of inhalation/exhalation onset/offset and manual marking of the events.
● Setting a threshold to define inhalation/exhalation onset/offset.

In some embodiments, (e.g. if the respiration variability is high) the hysteresis value is not constant for the whole session but is calculated using a sliding window of 30 seconds, based on the respiration variance.

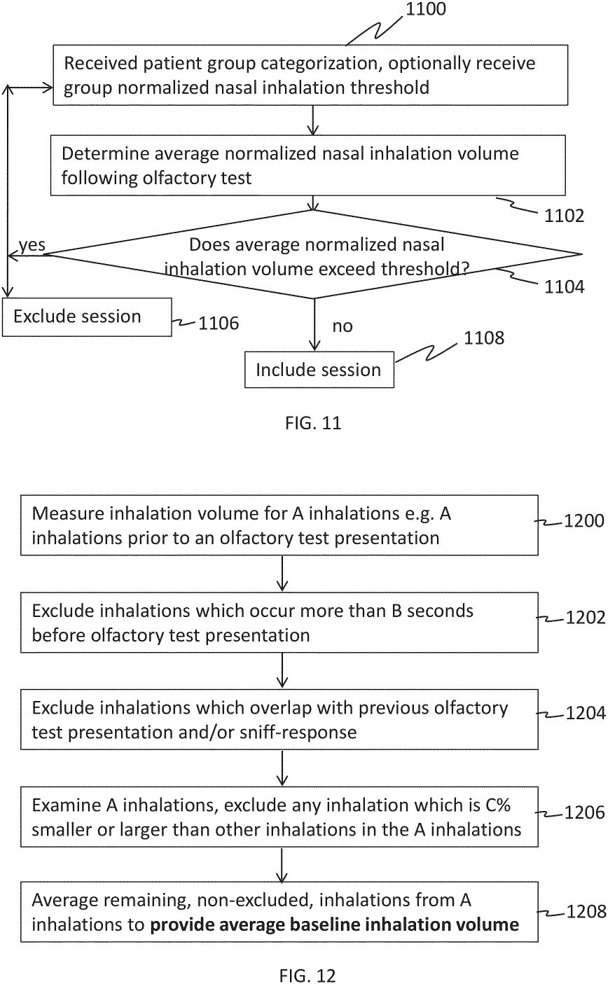

At 906, in some embodiments, data is evaluated and the session is included or excluded, based on the evaluation. Where session evaluation includes one or more feature as illustrated in and/or described regarding FIG. 10 and/or FIG. 11.

Referring to experimental results (e.g. as described elsewhere in this document). Exclusions for olfactory testing sessions were performed according to the exclusion criteria described in FIG. 10 and FIG. 11.

Out of 190 olfactory testing sessions, 41 sessions were excluded due to lack of stable nasal respiration, and 3 sessions were excluded due to nasal inhalation volume larger than 3.5 SD of the group mean, maintaining 146 sessions in 43 subjects.

146 sessions were conducted in total (1-12 sessions per subject, mean = 3.4 ± 3), with inter-session intervals ranging between 1 and 10 weeks (mean = 2.65 ± 1.7). In some embodiments, these intervals were considered to allow longitudinal comparison in 31 subjects.

Overall, 73 of the sessions were conducted in MCS (in 31 subjects), and 73 sessions in VS/UWS (in 24 subjects, 16 transitioned from VS/UWS to MCS during the study).

At 908, in some embodiments, data for one or more trial (e.g. each olfactory trial) is evaluated. In some embodiments, one or more trial (e.g. each trial) included or excluded, based on the evaluation. Where trial evaluation includes one or more feature as illustrated in and/or described regarding FIG. 13.

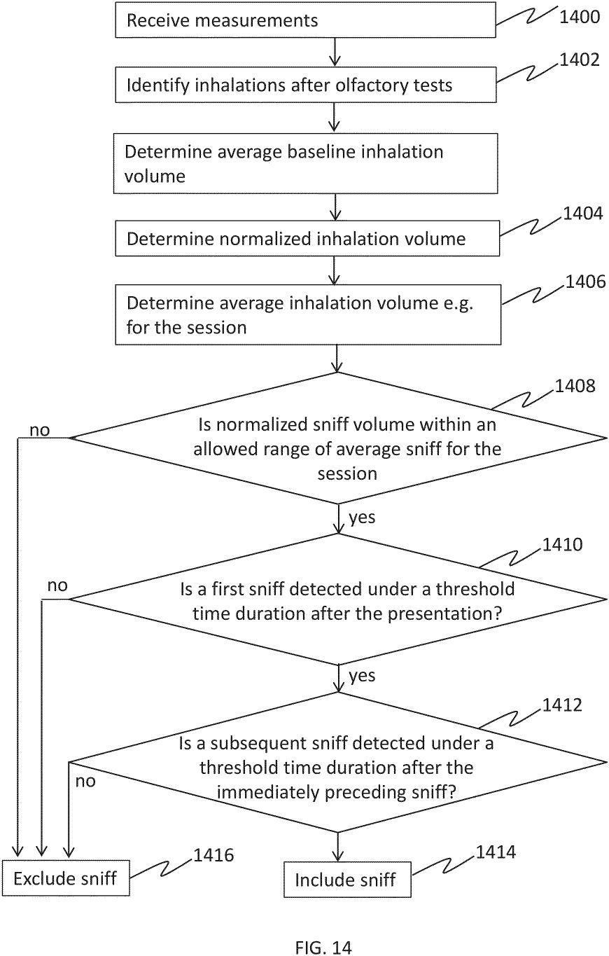

At 910, in some embodiments, sniffs are identified from the data e.g. where identification includes one or more feature as illustrated in and/or described regarding steps 1400-1406 FIG. 14.

At 912, in some embodiments, data for one or more sniff is evaluated (e.g. each sniff). In some embodiments, one or more sniff (e.g. each sniff) is included or excluded, based on the evaluation. Where sniff evaluation includes one or more feature as illustrated in and/or described regarding steps 1408-1416FIG. 14.

### Exemplary Olfactory Testing

FIG. 10 is a method of olfactory testing, according to some embodiments of the invention.

At 1000, in some embodiments, one or more sensor is positioned to measure nasal airflow of the subject and/or activated to initiate nasal airflow measurements. For example, where step 1000, in some embodiments, includes one or more feature as illustrated in and/or described regarding step 604FIG. 6B.

At 1002, in some embodiments, the subject’s nasal respiration is assessed e.g. whether the subject has stable respiration or not is verified. For example, in some embodiments, a user assessing a subject visually and/or aurally (e.g. stethoscope) verifies that the subject has stable respiration. In some embodiments, stable respiration is verified automatically. Where, for example, frequency and/or volume and/or variability of the respiration is measured and/or determined, using one or more of these parameters stable respiration is verified (or not verified). In some embodiments, one or more of these respiration parameters are compared with a threshold. In some embodiments, the threshold/s depend on one or more group and/or individual characteristic of the subject.

At 1004, in some embodiments, if the subject does not have stable nasal respiration, a time period is allowed to elapse. For example, in some embodiments, if no nasal respiration is observed and/or measured a time period of 1-10 minues is allowed to elapse.

At 1006, in some embodiments, after the time period has elapsed, the subject’s nasal respiration is assessed e.g. re-assessed.

At 1008, in some embodiments, if the subject does not have stable nasal respiration, the subject is repositioned. For example the subject is turned and/or partially and/or fully elevated and/or de-elevated e.g. by changing a position of a bed on which the subject is disposed.

In some embodiments, repositioning is automatic, e.g. by sending control signal/s to actuator/s configured to move the patient e.g. bed actuator/s.

In some embodiments, alternatively or alternatively to re-positioning, the patient is exposed to a stimulus. For example, an aural and/or visual and or olfactory stimulus.

At 1010, in some embodiments, the subject’s nasal respiration is assessed.

At 1012, in some embodiments, if the subject does not have stable nasal respiration (e.g. despite waiting and/or repositioning) the session is excluded and/or terminated. In some embodiments, termination and/or exclusion of a session is not an indicator that future sessions should not be attempted.

At 1014, in some embodiments, an olfactory trial is performed e.g. performance of the olfactory trial including one or more feature of olfactory trial as illustrated in and/or described regarding FIG. 4 and/or step 608FIG. 6B.

At 1016, in some embodiments, a number of trials performed is assessed.

At 1018, in some embodiments, if sufficient trials have been performed, the session is included and/or terminated. In some embodiments, a minimum number of trials is 2-30, or 10-20, or lower or higher or intermediate numbers of trials. In an exemplary embodiment, a minimum number of trials is 15.

At 1020, in some embodiments, if insufficient trials have been performed, optionally, nasal respiration of the subject is assessed. In some embodiments, if insufficient trials have been performed and, optionally, assessment of subject respiration indicates that the subject has stable nasal respiration, an additional olfactory trial is performed.

At 1022, in some embodiments, if insufficient trials have been performed and the subject does not have stable nasal respiration, the session is terminated and/or excluded. In some embodiments, before termination and/or exclusion steps 1004 and/or 1008 are performed before returning to step 120.

### Exemplary Inclusion/Exclusion of Data

### Exemplary Olfactory Session Inclusion/Exclusion

FIG. 11 is a method of session assessment, according to some embodiments of the invention.

At 1100, in some embodiments, a patient group characterization is received, for example, the characterization being provided by one or more clinical assessment e.g. clinical assessments described in step 610FIG. 6B.

At 1102, in some embodiments, average nasal inhalation volume following olfactory tests (e.g. average normalized inhalation volume) are determined.

At 1104, in some embodiments, the average normalized nasal inhalation volume is compared with a threshold. In some embodiments, the threshold is based on the patient group characterization. For example, in some embodiments, it is based on a group average.

Where, in some embodiments, the threshold was based on a subject group mean. For example, in some embodiments, the subject has previously been categorized (e.g. using clinical consciousness assessment/s) in a subject group. A group mean for the group is then determined and used to provide the threshold. In some embodiments, the threshold is 1-10, or 1-5, or 2-5 times the group mean, or lower or higher or intermediate ranges or multiples. In an exemplary embodiment the threshold is 3.5 SD of the group mean.

In some embodiments, the group mean is a value received from a database e.g. compiled from representative data. In some embodiments, the database value is updated as more data is collected.

At 1106, in some embodiments, if the average normalized nasal inhalation volume exceeds the threshold, the session is excluded e.g. from patient assessments.

At 1108, in some embodiments, if the average normalized nasal inhalation volume does not exceed the threshold, the session is included e.g. in patient assessment.

Referring to experimental results (e.g. as described elsewhere in this document) the inclusion/exclusion criteria as described in steps FIG. 11 excluded one session from each of three different subjects.

All of these three subjects had a later score of at least 48 (48/62/84) on the Functional Independence Measure (FIM)²⁸ and at least 49 (49/55/57) on the LCS⁵¹, indicating emergence from MCS. This suggests that, in some embodiments, nasal inhalation is considered to be informative of consciousness even in cases of potentially altered olfaction e.g. associated with to outlying respiration volume compared to group mean.

### Exemplary Determining of Averaged Baseline Inhalation Volume

FIG. 12 is a method of determining an average baseline inhalation volume, according to some embodiments of the invention.

At 1200, in some embodiments, respiration is measured for A inhalations prior to an olfactory test presentation. Where A is, for example, 3, or 1-10, or 1-5, or lower or higher or intermediate numbers or ranges.

At 1202, in some embodiments, inhalation/s which occur more than B seconds before the olfactory test presentation are excluded. Where B is, for example, 30, or 10-60, or lower or higher or intermediate numbers or ranges. Alternatively, or additionally, inhalation/s which occur more than B seconds before a sniff (e.g. a first sniff) are excluded.

At 1204, in some embodiments, inhalation/s which overlap with a previous olfactory test presentation and/or previous sniff response are excluded.

At 1206, in some embodiments, inhalation/s which are C% smaller or larger than the other A inhalations (e.g. an average of the other A inhalations and/or an average of the A inhalations) are excluded. Where C is, for example, 25, or 10-50, or 15-30, or lower or higher or intermediate numbers or ranges.

For example, in an exemplary embodiment, A is 3 and one of the three baseline inhalations is 25% smaller or larger than the other two baseline inhalations. In this case the baseline inhalation with the maximal difference in inhale volumes from the median volume of the three inhalations is excluded.

At 1208, in some embodiments, remaining inhalations (not excluded) of the A inhalations are averaged to provide an average baseline inhalation volume.

Referring to experimental results (e.g. as described elsewhere in this document) the inclusion/exclusion criteria as described in steps 1200-1208 retained 16,300 out of 17,334 baseline inhalations (i.e., 5.96% excluded). In some embodiments, the exclusion/inclusion criteria (e.g. of step 1200-1208) are adjusted to exclude a percentage of trials e.g. 0.05%-20%, or 1%-10%, or 4%-8%, or about 6%, or lower or higher or intermediate percentages or ranges.

### Exemplary Inclusion or Exclusion of an Olfactory Trial

FIG. 13 is a method of assessing an olfactory trial, according to some embodiments of the invention.

At 1300, in some embodiments, nasal inhalation data prior to and during a trial is received.

At 1302, optionally, in some embodiments, if baseline inhalation prior to a trial is unstable, the trial is excluded.

Where unstable baseline inhalation, in some embodiments, is where there is monatomic decrease or increase in volume of a number of inhalations preceding the trial.

Where, in an exemplary embodiment, for three inhalations proceeding the trial, monatomic decrease or increase is defined as an at least 40% difference between peaks of the 1^{st} and 3^{rd} baseline inhalations, and at least a 25% difference between the 1^{st} and 2^{nd}, and between the 2^{nd} and 3^{rd} inhalations.

At 1304, optionally, in some embodiments, if insufficient baseline inhalation is detected prior to stimulus presentation, the trial is excluded. For example, in some embodiments, if respiration has values which are too low e.g. respiration is not detected automatically by filtering and/or application of hysteresis. For example, where less than the required number (e.g. 3) of inhalations are detected prior to the stimulus presentation (e.g. less than the required number of inhalations between presentations).

At 1306, optionally, in some embodiments, if no inhalation (no sniff) is detected within 6.5 seconds after the olfactory test presentation the trial is excluded.

At 1308, optionally, in some embodiments, if a difference between the baseline inhalation and sniff volume is outside a threshold, the trial is excluded. For example, in some embodiments a trial is excluded if there is one or more of:

An extreme change in the inhalation volume between baseline and the following inhalations.

In some embodiments, standard deviation and mean are calculated for baseline inhalation volume σ_{b1}, µ_{b1}, and for inhalations after the presentation of a stimulus δ_{sniff}, µ_{sniff●}

In some embodiments, the coefficient of variation (CV) (a ratio of standard deviation to mean) is then calculated for both sets of inhalations: CV_{b1}= σ_{b1}/µ_{b1}, CV_{sniff}= σ_{sniff}/µ_{sniff}

In some embodiments, a trial is excluded if one or more of:
A maximum of the two CVs; CV_{b1} and CV_{sniff} is below 20%: CV_{b1} < 20% and CV_{sniff} < 20%
A percent signal change (PSC) of the two CVs is below 50%: CV_{sniff} /CV_{b1} X 100% < 50%
A PCS of the two means is above 50%: µsniff /µb1 x100% <20%

Referring to exemplary experimental results (e.g. described in more detail elsewhere in this document), the exclusion/inclusion criteria of 1308 was applied and 12 trials corresponding to 0.19% of all trials were excluded. In some embodiments, the exclusion/inclusion criteria (e.g. of step 1308) is adjusted to exclude a percentage of trials e.g. 0.01%-10%, or 0.01%-1%, or 0.01%-0.5%, or 0.1%-0.3%, or lower or higher or intermediate percentages or ranges.

At 1310, Referring to exemplary experimental results (e.g. described in more detail elsewhere in this document) trial exclusion/inclusion criteria as described in steps 1302-1310 were applied and retained 5,778 out of 5,934 trials remaining after session exclusion (i.e., 2.63% excluded). In some embodiments, the exclusion/inclusion criteria (e.g. of one or more of steps 1302-1308) is adjusted to exclude a percentage of trials e.g. 0.5%-10%, or 1%-5%, or lower or higher or intermediate percentages or ranges.

### Exemplary Inclusion or Exclusion of Sniffs

FIG. 14 is a method of assessing sniff inclusion, according to some embodiments of the invention.

At 1400, in some embodiments, nasal inhalation data during an olfactory testing session is received.

At 1402, in some embodiments, inhalations after olfactory tests are identified from the nasal inhalation data. In some embodiments, the inhalation/s which occur after a stimulus/stimuli are identified. In some embodiments, nasal airflow measurements are collected along with timing data of olfactory testing presentations to the subject. In some embodiments, those inhalations following a presentation are identified using this timing data.

At 1403, in some embodiments, an average baseline inhalation volume (ABIV) is determined.

In some embodiments, ABIV determination includes one or more feature as illustrated in and/or described regarding steps 1202-1208FIG. 12.

Where, in some embodiments, the term “baseline” refers to respiration where the subject is not being tested and/or stimulated.

In some embodiments, the ABIV is an average for an individual, where, for example, a plurality of non-stimulation inhalations for the individual are used, e.g. prior to a start of a session.

In some embodiments, the ABIV is an average for a group in which the individual has been categorized e.g. VS/UWS and/or any other types of patient group described in this document.

In some embodiments, the ABIV is updated periodically, for example, continuously within a testing session e.g. to account for temporal variation in inhalation volumes. For example, in an exemplary embodiment, the baseline inhalation is an average of three inhalations prior to performance of an olfactory test presentation. In some embodiments, one or more baseline inhalation is excluded from an ABIV.

In some embodiments, nasal inhalation/s following a stimulus (also herein termed “sniff/s” are normalized, e.g. by dividing the sniff volume a baseline inhalation volume. Potentially, normalizing inhalations e.g. by dividing by a baseline inhalation volume reduces effect of changes in respiration pattern across a session and/or between sessions on inhalation data collected.

At 1404, in some embodiments, inhalations after olfactory tests (sniff volumes) are normalized. In some embodiments, a sniff volume is normalized by dividing the sniff by a baseline inhalation volume. Where the baseline inhalation volume is, in some embodiments, determined using one or more feature as described and/or illustrated in FIG. 12. For example, in some embodiments, each sniff is normalized by dividing the sniff volume by a baseline inhalation volume. Potentially, normalizing using an average of baseline inhalation reduces effect of changes in respiration pattern across an olfactory testing session and/or between sessions.

In some embodiments, alternatively or additionally to normalizing using an averaged baseline inhalation volume (calculation of e.g. as in FIG. 12), a coefficient of variation (CV) for respiration is used to normalized the respiration measurement/s. Where CV is a standardized measure of dispersion of a probability distribution or frequency distribution e.g. the ratio of the standard deviation to the mean.

In some embodiments, normalizing is additionally or alternatively using baseline averages not from the subject. For example, a baseline average determined using subject data (e.g. one or more of age, sex, assumed consciousness level) and/or based on healthy subjects and/or for a subject in a higher level of consciousness (e.g. one stage higher) e.g. a baseline average value from MCS subject/s in order to normalize data from an VS/UWS subject.

At 1406, in some embodiments, an average inhalation volume e.g. for the olfactory testing session is determined.

In some embodiments, a sniff is included e.g. in patient assessment, if one or more of (or all of):

At 1408, the normalized sniff inhalation volume is within an allowed range. For example, an allowed range outside the average inhalation volume after olfactory testing. In an exemplary embodiment the allowed range is ± 3.5 SD of the averaged sniff in the session.

At 1410, in some embodiments, a sniff is included if the sniff occurs within a time duration after the olfactory presentation. In some embodiments, the time duration is a standard time for all subjects e.g. 6.5 seconds. In some embodiments, if a first sniff is not detected within the time duration, subsequent sniffs of the olfactory trial are also excluded.

In some embodiments, the time duration is based on a respiration frequency of the subject and/or of the subject group. In some embodiments, the time duration is based on a multiple (e.g. double, or 1.5-5 times or lower or higher or intermediate ranges or values) of an averaged respiration cycle duration (e.g. between two inhales and/or between two exhales) for an individual.

For example, in the experiments described in the “Exemplary Results” section of this application, in 8 subjects, respiration rate was slower than typical and the threshold was increased to 7.5 seconds in 3 subjects, 8.5 seconds in 3 subjects, and 11 seconds in one subject.

At 1412, in some embodiments, for example, if subsequent (non-first) sniffs after an olfactory presentation are to be evaluated in assessment of a subject, a time duration between the subsequent sniff and an immediately previous sniff is evaluated. For example, a time duration between the second and first sniffs. For example, a time duration between the third and second sniffs. In some embodiments, the time duration is a standard time for all subjects e.g. 6.5 seconds, for example, successive sniffs are included if detected within 6.5 seconds of the previous sniff. Alternatively, in some embodiments, the time duration is based on a respiration frequency of the subject and/or of the subject group.

Referring to exemplary experimental results (e.g. described in more detail elsewhere in this document) sniff exclusion/inclusion criteria as described in steps 1402-1412 were applied and retained 16,999 out of 17,334 sniffs (i.e., 1.93% excluded). In some embodiments, the exclusion/inclusion criteria (e.g. of one or more of steps 1402-1312) is adjusted to exclude a percentage of trials e.g. 0.5%-10%, or 0.5%-5%, or 1-3%, or lower or higher or intermediate percentages or ranges.

### Exemplary Experimental Results

### Exemplary Subjects

Fifty subjects (mean age 43.4 ± 17 years, 9 women), who had suffered brain injury and were suffering from a DoC, were recruited across a span of ~4 years at the Intensive Care and Rehabilitation of Consciousness Department at the Loewenstein rehabilitation hospital, Ra’anana, Israel.

### Exemplary Data Collection

Subjects are tested with multiple olfactory testing sessions (range 1-13 sessions, mean 3.8 ± 2.98 sessions, total 190 sessions) separated by days/weeks depending on the individual subject clinical and personal availability.

### Exemplary VS/UWS Results

9 of 24 VS/UWS subjects had an odorant detection sniff response in at least one session. Sniff responses as defined by normalized sniff volume threshold of 15% and sniff volume variability threshold of 3.5 SD.

Of these, one subject had an odorant differentiation sniff response as well. Where the odorant differentiation sniff response was defined at least a 20% difference in normalized sniff volume between different odorants and where both odorants are lower in volume than baseline.

Cognitively-driven sniff responses were observed in nine VS/UWS subjects (where sniff response thresholds were the same as odorant detection sniff thresholds), eight of which had an odorant detection sniff response (the ninth with only a trend).

Although, results failed to indicate a sniff response in VS/UWS sessions at the group level, 10 of 24 individual VS/UWS subjects had sniff responses in at least one session.

Clinical progression over time was assessed for VS/UWS subjects with sniff responses and all 10 VS/UWS subjects who had a sniff response in one session or more, later transitioned into MCS.

Thus, according to the sniff response thresholds used, a sniff response in VS/UWS indicated transition to MCS at 100% specificity and 62.5% sensitivity (10 out of 16 VS/UWS subjects who transitioned), indicating that, in some embodiments, sniff responses are informative for prognosis at the single subject level.

In order to exclude bias introduced by the possibility that those subjects who transitioned from VS/UWS to MCS live longer and thus having a greater probability of participating in repeated sessions, the number of sessions per participant were compared, and no significant difference in the number of sessions between VS/UWS subjects who transitioned to MCS and those who remained in VS/UWS was observed:
● overall number of sessions:
   transitioned = 5.9 ± 3.5
   non-transitioned = 4 ± 2.6, t(22) = 1.33, p = 0.198
● sessions included:
   transitioned = 5.1 ± 3.7
   non-transitioned = 3.1 ± 3.0, t(22) = 1.3, p = 0.21)

In four of these subjects the sniff response preceded any other sign of consciousness recovery by days up to months: 2.5 months, ~2 months, -1.5 months, 2 days.

### Exemplary MCS Results

20 of 31 MCS subjects had sensory driven sniff responses, in at least one session. Were sniff responses were defined by normalized sniff volume threshold of 15% and sniff volume variability threshold of 3.5 SD.

In at least one session, 19 subjects had odorant detection sniff responses, 13 of these subjects had cognitively-driven sniff responses, and 4 of these subjects had scent differentiation sniff responses. One subject had only a strong trend towards odorant detection sniff response and a significant cognitively-driven sniff response.

This implies sensitivity of 64.5% in determining MCS using sniff response criteria measure.

The false negative rate reflects that ~35% of MCS subjects had no sniff response, indicating that a lack of sniff responses does not necessarily imply unconsciousness. For example, in some embodiments, a lack of sniff response is used to assess olfaction-related brain structure injury. For example, in some embodiments, sniff response is used to evaluate subjects where olfaction-related brain structure injury has been excluded.

### Exemplary Sniff Response to Differentiate Between VS/UWS and MCS

Results indicated that sensory sniff responses are evident both in MCS vs. VS/UWS sessions, and in MCS vs. VS/UWS subjects.

Analysis by session is first described: Normalized nasal inhalation volume of the first three nasal inhalations following unpleasant and pleasant odorants vs. respiratory baseline in MCS and VS/UWS sessions was compared.

In some embodiments, e.g. in response to abnormal distribution of the data in both MCS and VS/UWS sessions and greater variance in MCS and in VS/UWS sessions non-parametric tests, Bonferroni correction was used for multiple comparisons.

At the group level, for the first sniff, MCS sessions had odorant detection sniff response and VS/UWS sessions did not have odorant detection sniff response.

Sniff responses significantly discriminated between VS/UWS and MCS states at the group level (p < 0.0001, effect-size r = 0.63).

Odorant detection sniff response evident in MCS sessions significantly differentiated them as a group from VS/UWS sessions. Similar results were evident in the second sniff after odorant presentation, but not in the third sniff.

In some embodiments, altered sniffing persisting into the second sniff after each odorant presentation but not to the third, is considered to indicate that the phenomena is a genuine transient odorant-driven response and not a state-change.

Comparing the odorant-driven sniff response to the first inhalation following Blank rather than to the respiratory baseline, produced similar results indicating sensory sniff responses in MCS but not VS/UWS sessions: The sniff response to odor is larger than the response to a blank presentation. The response to odors was observed in the 1st and 2nd sniff following odor onset and for blank only in the 1st sniff.

Analysis by subject is now described.

Data was analyzed only considering the single strongest sniff response session from each participant who remained unchanged throughout the study. Selecting the strongest session is also consistent with the standard approach in DoC where in behavioral assessment (CRS-R), recommendation is to select the highest score of at least five sessions²².

This analysis retained 19 MCS subjects and 8 VS/UWS subjects.

Using the strongest session from each individual subject:
● A significant odorant-induced sniff response was observed in MCS subjects:
   Sniff 1: Pleasant = 0.831 ± 0.18 NFU, median = 0.89 NFU, Unpleasant = 0.829 ± 0.21 NFU, median = 0.93 NFU, difference from baseline, all Z > 2.9, all p < 0.004, Bonferroni corrected, all effect-size r > 0.66,
● And no odorant-induced sniff response was observed in VS/UWS subjects
   Sniff 1: Pleasant = 0.97 ± 0.11 NFU, median = 0.92 NFU, Unpleasant = 0.94 ± 0.06 NFU, median = 0.96 NFU, difference from baseline, all p > 0.05, Bonferroni corrected).

It was observed that MCS patients were not only significantly different from baseline, they were also significantly different from the average VS/UWS value (Sniff 1 across odorants: Z = 2.2, p = 0.03, effect-size r = 0.50). Thus, sensory sniff-responses were evident not only in MCS vs. VS/UWS sessions, but also in MCS vs. VS/UWS patients.

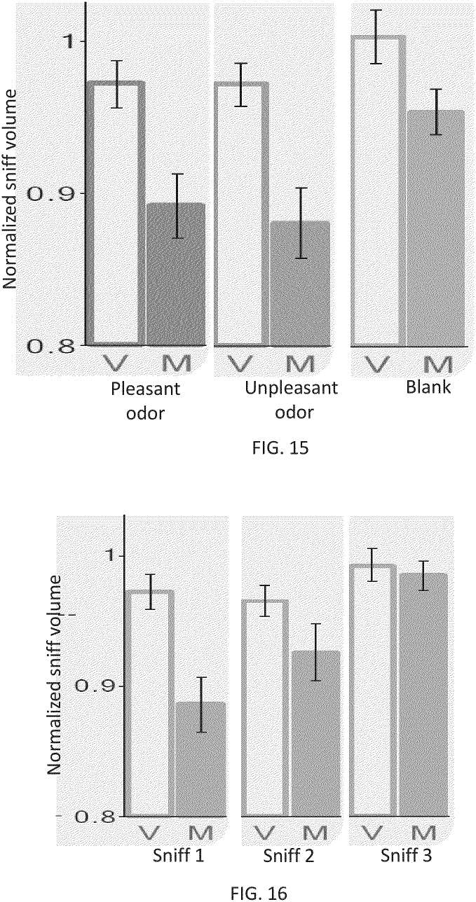

FIG. 15 is a simplified schematic of normalized sniff volume for different olfactory tests and different subject types, according to some embodiments of the invention.

FIG. 16 is a simplified schematic of normalized sniff volume for different subject types for successive sniffs after olfactory test presentation, according to some embodiments of the invention.

In FIG. 15 and FIG. 16 “V” indicates VS/UWS subjects and “M” indicates MCS subjects as categorized using clinical consciousness assessment/s.

Apparent from FIG. 15 is that, at the group level, subjects with different states of consciousness have different normalized sniff volume, for all types of olfactory test.

Apparent from FIG. 16 is that, at the group level, difference in normalized sniff volume between patient groups extends to the second sniff following an olfactory test.

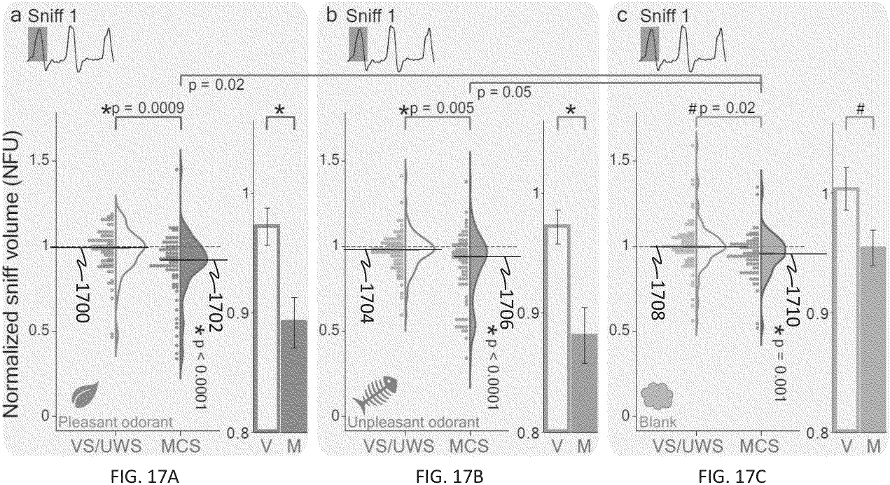

FIGS. 17A-C are simplified schematics showing results of different types of olfactory tests, for the first sniff after the trial, for VS/UWS subjects and MCS subjects, according to some embodiments of the invention.

In some embodiments FIGS. 17A-C illustrate details of results leading to results illustrated in FIG. 15. Where FIG. 17A shows normalized sniff volume following pleasant odorants, FIG. 17B shows normalized sniff volume following unpleasant odorants, FIG. 17C shows normalized sniff volume following blank presentations.

In some embodiments, each dot represents a session, flat violin plots show the distribution, lines 1700, 1702, 1704, 1706, 1708, 1710 denote medians. The y-axis “1 value” is indicated with a dashed horizontal line to denote the baseline value at 1 normalized flow units (NFU).

The bar-graphs (also illustrated separately in FIG. 15) to the right of each distribution tabulate the same data, with error bars denoting standard error of the mean (SEM).

The p-value beneath the distribution denotes its difference from baseline inhalation, i.e., the existence of a sniff response e.g. the likelihood the outcome is chance as opposed to being significant. For example, in some embodiments, a p-value smaller than 0.05 denotes a significant reduction in sniff volume in response to odor/blank.

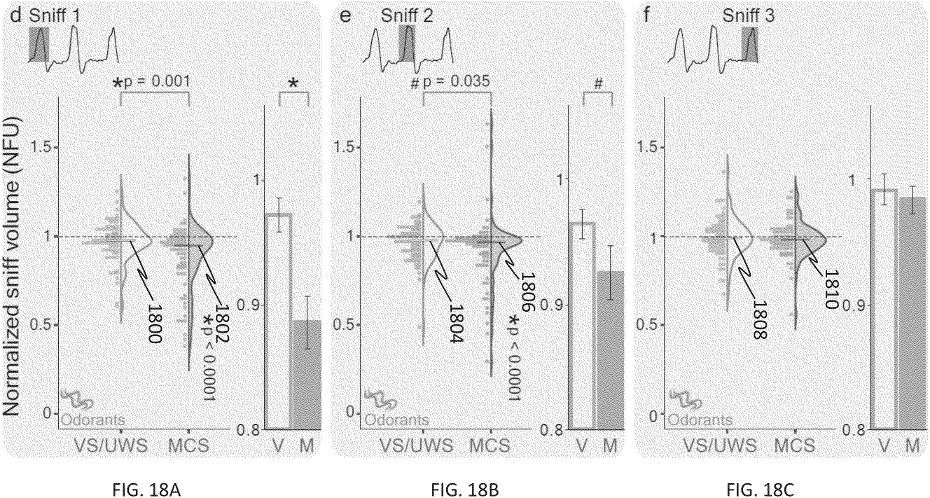

FIGS. 18A-C are simplified schematics showing results of olfactory testing, for the first three sniffs after a trial, for VS/UWS subjects and MCS subjects, according to some embodiments of the invention.

In some embodiments FIGS. 18A-C illustrated details of results leading to results illustrated in FIG. 16.

FIG. 18A, FIG. 18B, FIG. 18C, show normalized sniff volume for the first, second and third sniffs after an olfactory trial, respectively.

In some embodiments, each dot represents a session, flat violin plots show the distribution, lines 1800, 1802, 1804, 1806, 1808, 1810 denote medians. The y-axis 1 value is indicated with a dashed horizontal line to denote the baseline value at 1 normalized flow units (NFU).

The bar-graphs (also illustrated separately in FIG. 16) to the right of each distribution tabulate the same data, with error bars denoting standard error of the mean (SEM).

The p-value beneath the distribution denotes its difference from baseline inhalation, i.e., the existence of a sniff response e.g. the likelihood the outcome is chance as opposed to being significant. For example, in some embodiments, a p value smaller than 0.05 denotes a significant reduction in sniff volume in response to odor/blank.

FIG. 18A is the data from FIG. 17A and FIG. 17B combined.

FIG. 18B is combined data for odorant (not blank) presentations, for the second sniff after the odor presentation.

FIG. 18C is combined data for odorant (not blank) presentations, for the third sniff after the odor presentation.

### Exemplary Cognitive Sniff Response

At the group level, for MCS sessions, nasal inhalation volume was significantly reduced in response to Blank presentations and for VS/UWS sessions was uninfluenced by Blank presentations: At the group level, a ~5% reduction in nasal airflow in response to Blank was observed MCS sessions. At the group level, no such reduction was observed in VS/UWS sessions.

In some embodiments, the cognitively-driven sniff response evident in MCS sessions significantly differentiates the MCS sessions from the VS/UWS sessions e.g. for the first sniff.

Experimental results indicated that this outcome is not evident in the second and third sniffs, indicating, in some embodiments, that this group difference is a transient task-driven response and not a state-change.

Results indicate that a cognitively-driven component of the sniff response reflects state of consciousness in DoC subjects, at least at the group level and, in some embodiments, is used to assess individual subjects.

### Exemplary Behavioral Clinical Evaluation

During experimentation, subjects were classified into groups (VS/UWS or MCS) using clinical consciousness assessment/s:

Twenty-one subjects were evaluated using CNC and LCS only, and the remaining subjects’ state of consciousness was evaluated using CRS-R, CNC, and LCS. In the cases where both CRS-R and CNC were used, level of consciousness is determined by the CRS-R.

To estimate the impact of having CNC and LCS estimates only, classification of VS/UWS and MCS between CRS-R and CNC scales was compared.

Disagreement was observed in 30 of 80 sessions with both scales:
29 sessions CRS-R indicated VS/UWS CNC suggested MCS
1 session CRS-R indicated MCS CNC suggested emergence from MCS

This could suggest that the CNC division to VS/UWS (‘extreme coma’, ‘marked coma’) and MCS (‘moderate coma’, near coma’) by the scale subcategories might be too liberal.

Out of the 21 subjects assessed only with CNC scale following the olfactory session, 4 were in VS/UWS in all sessions, 12 were in MCS in all sessions, and 5 transitioned between VS/UWS and MCS across sessions.

If indeed the CNC is too liberal, it is possible that some MCS subjects were in fact VS/UWS. The consequences of such misclassification on the group level analysis means that we could have only underestimated the observed findings. Thus, we conclude that although such misclassification would be unfortunate, it would not weaken, but only strengthen our effects.

A second possible consequence of the use of CNC scale is detection of recovery while no recovery occurred.

Out of the 16 subjects who transited from VS/UWS to MCS, 5 were assessed using the CNC but not with the CRS-R.

Importantly, in all five subjects the Loewenstein communication scale (LCS) conducted independently by the hospital team provided additional evidence for conscious awareness in all of the five subjects, and supported the CNC behavioral assessment. Thus, the lower sensitivity of CNC versus CRS-R in this study may have underestimated the power of the results, but does not appear to inaccurately detect transition to MCS.

### Exemplary Results for Exemplary Subjects

Discussion of exemplary subjects, in some embodiments, illustrates advantages of assessing individual sessions for subjects, e.g. given the possibility of fluctuating and/or changing consciousness levels.

Subject #4 started the study with a sniff response session in MCS, then deteriorated, conducting his following session in VS/UWS, only to later recover, and conduct a third and final sniff response session again in MCS (today Subject #4 walks and talks).

In some embodiments, averages of different sessions are used to provide an average subject assessment. Alternatively or additionally, in some embodiments, data is analyzed “by session” and not “by patient”.

Subject #6 had 5 sessions, 4 in VS/UWS and one in MCS, yet he had a sniff response in only one of these sessions, the VS/UWS session directly before transitioning. This too would be obscured by a nested design.

### Exemplary VS/UWS Subject Recovery Prediction

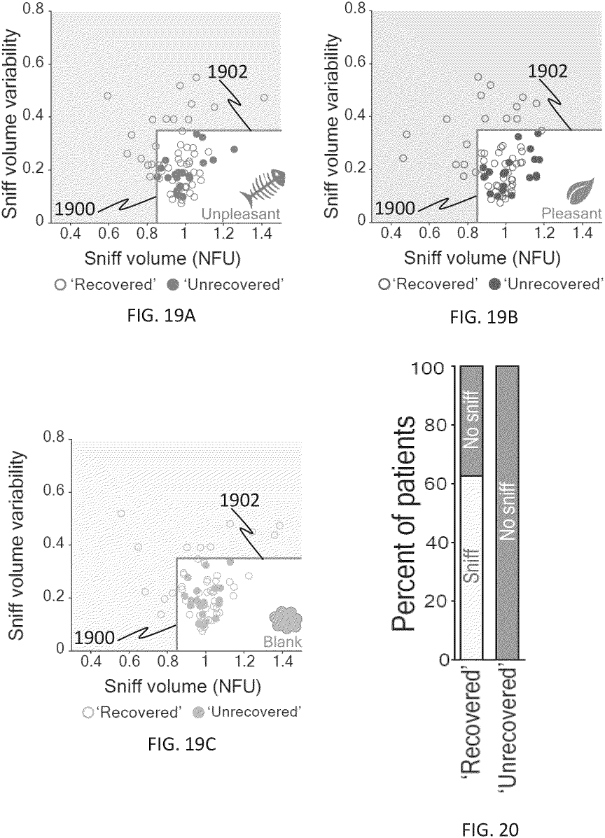

FIGS. 19A-C are simplified schematics of sniff volume variability with sniff volume, for different olfactory tests, for VS/UWS subjects, according to some embodiments of the invention.

Illustrated on FIGS. 19A-C are a sniff volume variability threshold 1900 and a normalized sniff volume threshold 1902, according to some embodiments of the invention. Where data points within both thresholds (under 3.5 SD variability across trials and over 0.85 normalized sniff volume) are designated sessions where the subject had a sniff response.

In FIGS. 19A-C, empty dots represent sessions in later “recovered” patients. Where “recovered” (also for FIG. 20), in some embodiments, indicates transfer to a higher level of consciousness, e.g. MCS (or even full consciousness) and “unrecovered” indicates that the subject remained in VS/UWS FIG. 20 is a simplified schematic of subject outcome and sniff response, for VS/UWS subjects, according to some embodiments of the invention.

### Exemplary DoC Subject Survival Prediction

Experimental results indicated that olfactory sniff responses predicted long-term survival rates for DoC subjects at 92% accuracy (X²= 14.5, p = 0.0001, Cramer’s V effect size = 0.45).

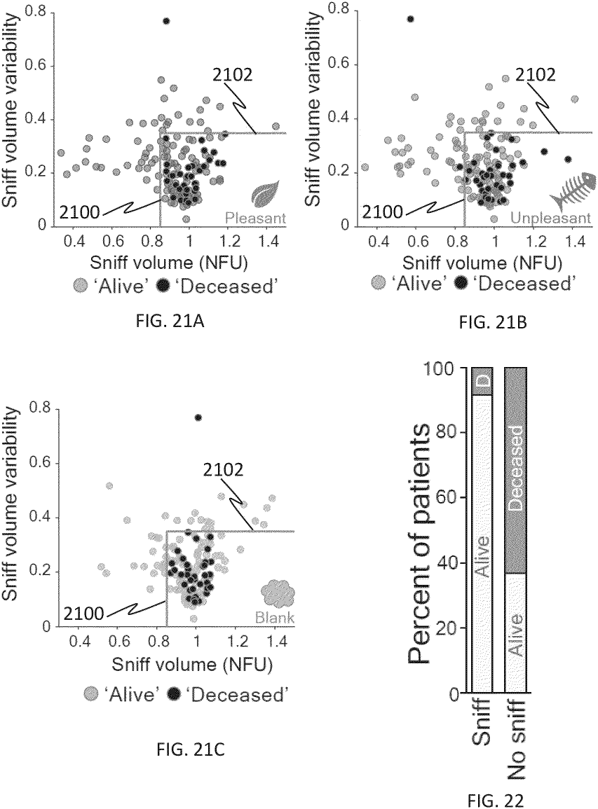

FIGS. 21A-C are simplified schematics of sniff volume variability with sniff volume, for different olfactory tests, for DoC subjects, according to some embodiments of the invention.

Illustrated on FIGS. 21A-C are a sniff volume variability threshold 2100 and a normalized sniff volume threshold 2102, according to some embodiments of the invention.

Where, in some embodiments, sniff volume threshold 2102 is sniffing with a more than 15% change in magnitude from baseline and, in some embodiments, sniff volume variability threshold 2100, of less than 0.35 SD.

In FIGS. 21A-C each dot is a DoC session (dots for both MCS and VS/UWS subjects) where black filled dots represent sessions in later deceased subjects and other dots represent sessions in surviving subjects (37.3 ± 14.1 months after brain injury).

FIG. 22 is a simplified schematic of subject outcome and sniff response, for DoC subjects, according to some embodiments of the invention.

FIG. 22 illustrates a percentage of DoC subjects (not sessions) with sniff responses (left) that survived (91.7%) or died (D) (8.3%), and of DoC subjects without sniff responses that survived (36.8%) or died (63.2%).

### Exemplary Sniff Response as a Predictor for Subject Functionality

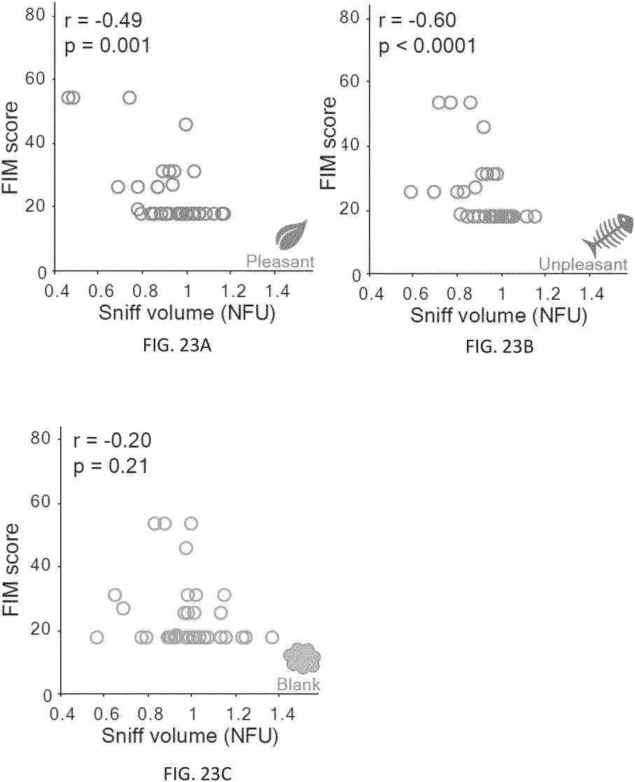

FIGS. 23A-C are simplified schematics of Functional Independence Measure (FIM) score with normalized sniff volume, for different olfactory tests, according to some embodiments of the invention.

In FIGS. 23A-C each dot illustrates sniff measurement results for a session where a subject was in VS/UWS, in a surviving patient. Where the FIM scores were determined after a time duration after injury for the subject, of on average across subjects = 37.4 ± 14.7 (range 17 -64) months after injury.

Subjects who had a sniff response shortly after injury were observed, mostly, to survive for years, whereas subjects who did not have a sniff response after injury mostly failed to survive during this period. More specifically, only two of 24 subjects (8.3%) who had a sniff response following their injury, then did not survive (died at 5 and 7 months after injury). The remaining 22 of 24 subjects (91.7%) with a sniff response following injury, currently survive (current average 37.3 ± 14.1 months after injury). In contrast, 12 of 19 subjects (63.2%) who did not have a sniff response following injury, then did not survive (died within 17.5 ± 12.2 months from injury, median = 13 months).

Sensitivity of the sniff response in predicting survival at 37.3 ± 14.1 months after brain injury is 91.7% (chi-square = 14.5, p = 0.0001, Cramer’s V effect size = 0.45).

Functional independence in the 29 surviving subjects were assessed using the Functional Independence Measure (FIM)²⁸. FIM was independently obtained in clinical testing of these subjects conducted 20.4 ± 11.5 months after injury. A significant correlation was observed, where an extent of sensory (but not cognitive) sniff response predicted later level of independence in VS/UWS subjects (Pleasant r₃₉_{Spearman} = -0.49, p = 0.001; Unpleasant r₃₉_{Spearman} = -0.60, p < 0.0001; Blank r₃₉_{Spearman} = -0.20, p = 0.21.

### Exemplary Investigation of Exemplary Sources of Variance

Three explorations of potential sources of variance in results were performed. Effect of trigeminal nerve-ending interference, tracheotomy and traumatic brain injury.

### Exemplary Trigeminal Interference

Without wanting to be bound by theory, in some embodiments it is assumed that, odorants can activate both olfactory and/or trigeminal nerve-endings in the nose. Potential trigeminal effect of use of odorant blends used were investigated and/or the effect of using a threshold determined from healthy people using pure odorants. To estimate whether the effects we observed depended on trigeminal contribution and/or if it is appropriate to use a threshold based on pure odorants for measurements collected using odorant mixes, pure olfactory odorants were used in a subset of subjects, and effects were replicated.

### Exemplary Effect of Tracheostomy

DoC subjects often breathe through a tracheostomy tube. It was investigated if tracheostomy modulated results. It was observed that, generally, although tracheostomy indeed significantly reduces nasal airflow, a measurable portion (e.g. according to embodiments described in this document) of nasal airflow remains. Exemplary results showed that although magnitude of nasal inhalation (not normalized) was significantly reduced, modulate normalized sniff responses were not affected. About 60% of experimental results MCS sessions and 80% of VS/UWS sessions were conducted in subjects with a tracheostomy (balloonless tracheostomy).

A 3-fold greater nasal inhalation volume in MCS sessions without tracheostomy was observed compared to sessions with tracheostomy (without = 0.07 ± 0.044, with = 0.02 ± 0.016, Z = 5.8 p < 0.0001, Cliff’s delta effect size = 0.8).

However, in some embodiments, sniff response is individually determined using individual baseline nasal airflow for normalization. Thus, in some embodiments, even low total levels of flow produce equal size sniff responses e.g. to those found in non-tracheostomy subjects.

In some embodiments, tracheostomy does not affect sniff results on the individual level. For example, results for the normalized sniff response in MCS sessions in which individual subjects were tested with and without tracheostomy did were similar:
A odorant detection sniff response was evident in the first and second sniff with tracheostomy:
all Z > 2.3, all p < 0.02, all r > 0.35 and without tracheostomy:
all Z > 2.93, all p < 0.003, all r > 0.54.

There were no significant differences in normalized sniff response magnitude between MCS sessions with and without tracheostomy:
all z < 1.7, all p > 0.09, all r < 0.31.

Results in MCS sessions, in some embodiments, are considered to imply that the difference observed in normalized sniff response between MCS and VS/UWS sessions is not be explained merely by different prevalence of tracheostomy between the MCS and VS/UWS groups (62% vs 84% respectively).

### Exemplary Impact of Traumatic Brain Injury on Olfaction

Many subjects of the exemplary experimental results suffered from traumatic brain injuries (TBI). Without wanting to be bound by theory, generally, it is considered that TBI can impact olfaction.

Indeed, structural brain imaging data revealed that 7 of the 8 subjects who had at least one session in MCS but never had a sniff response, had damage to olfaction-related brain structures.

If TBI is not equally distributed across the subject subgroups, this may have biased results.

To address this, a proportion of TBI in each subject group was compared of subject groups; MCS subjects, subjects who transitioned between VS/UWS and MCS and VS/UWS subjects, and no differences were observed.

Reanalysis of non-TBI participants alone generated a similar outcome.

### Exemplary Statistical Analysis

Normalized nasal inhalation volume values are not normally distributed and display greater variance in MCS than in VS/UWS sessions:

| | | |
|---|---|---|
| | Shapiro-Wilk test | Leven’s test |
| First Sniff | all W’s > 0.88, all p’s < 0.0007 | F(1,147) = 3.9, p = 0.05 |
| Second Sniff | all W’s > 0.66, all p’s < 0.01 | F(1,147) = 3.3, p = 0.07 |
| Third Sniff | all W’s > 0.63, all p’s < 0.04 | F(1,147) = 1.6, p = 0.21) |

In some embodiments, nonparametric tests are applied.

For statistical analysis between MCS and VS/UWS sessions Wilcoxon ranksum tests were used.

For statistical analysis within each group Wilcoxon signrank tests were used.

Bonferroni correction for multiple comparison is applied for comparison of nasal inhalation volume between MCS and VS/UWS sessions and also within a group (two odors X three sniffs: 0.05/6 = 0.0083).

Nonparametric dependent samples effect size is calculated by the following formula r = Z/sqrt(n)⁵³, where Z is the Wilcoxon signrank statistic and n is the sample size.

The nonparametric independent samples effect size is estimated using cliff’s delta⁵⁴. Chi-square effect size is estimated using Cramer’s V^{SS}.

Relation between the sniff responses and Functional Independence Measure (FIM)²⁸ are assessed using Spearman correlations and are Bonferroni corrected for multiple comparisons [two states X three sniffs: 0.05/6 = 0.0083].

Note: three sessions of two subjects who presented outliers sniff response value in later sessions implying impaired olfaction are excluded from the correlation analysis.

When including these three sessions similar results obtained: (Pleasant r₄₂ = -0.27, p = 0.075; Unpleasant r₄₂ = -0.45, p = 0.002; Blank r₄₂ = -0.04, p = 0.80).

### Exemplary Respiration Features and Parameters

FIG. 25 is a simplified schematic trace 2500 of measurement of respiration, according to some embodiments of the invention.

In some embodiments, trace 2500 is of a measurement of airflow with time for a single breath.

FIG. 25, in some embodiments, illustrates exemplary respiration features including; the respiration trace itself (where variability of the respiration trace itself is, in some embodiments, a respiration parameter), exhalation duration 2502, exhalation peak 2504, exhalation volume 2506 (area under respiration trace 2500), inhalation duration 2508, inhalation peak 2510, inhalation volume 2512 (area under respiration trace 2500). duration of a breath = exhalation duration 2502 + inhalation duration 2508.

In some embodiments, exemplary respiration parameters include averages of respiration features and/or variability of respiration features, for one or more time period.

### Exemplary Experimental Results for Non-Olfactory Respiration Parameters

FIG. 26 illustrates statistical significance of exemplary respiration parameters for subject assessment, according to some embodiments of the invention.

The subjects and data collected as described in the sections of this document entitled “Exemplary subjects” and “Exemplary data collection” were used to determine respiration parameters. For example, the respiration parameter/s as illustrated in and/or described regarding FIG. 25.

Using timing of presentation of stimuli (olfactory or blank presentation) non-sniff response respiration was used to determine non-olfactory respiration parameters. Where, in determination of respiration parameters, three respirations after a presentation were removed from respiration. For example, associated with the assumption that a presentation affects up to the third inhalation after the presentation.

FIG. 26 illustrates p-values of statistical significance of the ability of listed respiration parameters to differentiate between state, recovery, and survival as indicated in the columns of the table of FIG. 26.

Parameters surrounded by a heavier box are those with p-values of less than 0.05 indicating statistical significance, in some embodiments.

Std in the table of FIG. 26 indicates “standard deviation”.

In some embodiments, one or more of the respiration parameters as indicated as statistically significant in FIG. 26 are used to determine one or more of state, likelihood of recovery, likelihood of survival.

In some embodiments, a single respiration parameter is used to determine all of state, likelihood of recovery, likelihood of survival. For example, duration of inhalation or exhalation.

Alternatively or additionally, other parameters are used, for one or more of state, likelihood of recovery, likelihood of survival.

For example, in some embodiments, duration of inhalation and/or exhalation is used to determine state and additional respiration parameters (e.g. respiration parameters indicated as being statistically significant in FIG. 26) are used to determine likelihood of recovery and/or likelihood of survival.

### Exemplary Classifier

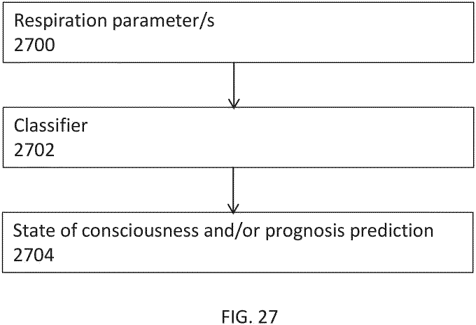

FIG. 27 is a simplified schematic block diagram, according to some embodiments of the invention.

In some embodiments, feature/s of the block diagram of FIG. 27 is performed by a processor and/or processing application, for example, processing application 218FIG. 2.

In some embodiments, measurement data is used to construct a classifier. For example, using sniff response data and/or respiration parameter data.

For example, in some embodiments, logistic regression classifier is constructed based on all or subset of respiration parameters described in this document, including non-sniff respiration parameters, odor induced-sniffing features or a combination of both kinds of features. Where, in some embodiments, the classifier is constructed by choosing a cutoff value and classifying inputs with probability greater than the cutoff as one class, below the cutoff as the other. In some embodiments, the classifier detects a consciousness state (e.g. VS/UWS vs. MCS) and/or predicts consciousness recovery and/or predicts survival.

For example, in some embodiments, a classifier is constructed using an alternative machine learning technique. For example, one or more of Perceptron, Naive Bayes, Decision Tree, K-Nearest Neighbor, Artificial Neural Networks/Deep Learning, and Support Vector Machine.

In some embodiments, input/s 2700 to a classifier 2702 include respiration parameter/s 2700. For example, including non-sniff and sniff response respiration parameters for a subject. Where non-sniff respiration parameters are for one or more time period, where in some embodiments, the time periods are non-overlapping and in some embodiments, the time periods are overlapping.

Optionally, in some embodiments, inputs to the classifier include the subject’s state of health with respect to expected effect on the subject’s physiological breathing apparatus. For example, subjects having respiration related conditions e.g. asthma, emphysema, pneumonia and/or conditions likely to affect respiration e.g. heart disease, in some embodiments, are assessed using different respiration parameter/s and/or using a portion of classifier 2702 which has be generated using respiration parameter data for this type of subject.

For example, in some embodiments, (e.g. where respiration volume is likely to be affected by a medical condition of the subject) volume respiration parameters are normalized before use in assessment of the subject.

In some embodiments, output/s 2704 of classifier 2702 include a probability that the subject is in a particular state of consciousness, for one or more state of consciousness. In some embodiments, output/s of classifier 2702 include a probability that the subject will recover (e.g. to a higher state of consciousness) and/or a probability that the subject will survive e.g. for one or more time period.

In some embodiments, the classifier determines a probability that a subject is in a group using one or more respiration parameter including in some embodiments, only non-sniff parameters and, in some embodiments, both sniff and non-sniff parameters. Where, in some embodiments, different parameters are weighted by the classifier.

### General

It is expected that during the life of a patent maturing from this application many relevant methods of assessment of subjects with DoC will be developed and the scope of the terms assessment of consciousness and/or assessment of subjects with DoC is intended to include all such new technologies a priori.

As used herein the term “about” refers to ± 10%

The terms “comprises”, “comprising”, “includes”, “including”, “having” and their conjugates mean “including but not limited to”.

The term “consisting of″ means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application is specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of assessing a subject comprising:
measuring a plurality of respirations of said subject;
determining one or more respiration parameter from said plurality of respirations; and
determining, using said one or more respiration parameter, one or more of:
a state of consciousness of said subject; and
a prognosis of said subject.

2. The method according to claim 1, wherein said one or more respiration parameter includes a measure of variation of one or more feature of respiration of said subject, over said plurality of respirations.

3. The method according to claim 2, wherein said one or more feature of respiration comprises duration of one or more of inhalation, exhalation, and a single respiration.

4. (canceled)

5. The method according to claim 1, wherein said determining comprises determining a state of consciousness of said subject, using one or both of:
a measure of variation of an inhale duration; and
a measure of variation of an exhale duration.

6. The method according to claim 1, wherein said determining a state of consciousness comprises one or both of:
determining a probability that said subject is in a minimally conscious state; and
determining a probability that said subject is in a vegetative state.

7. The method according to claim 1, wherein said determining a prognosis comprises determining one or more of:
a probability of recovery of said subject; and
a probability of survival of said subject.

8. The method according to claim 7, wherein at least one of:
said recovery includes transferring from a lower to a higher state of consciousness, over a time period of one month to 1 year from respiration measurement; and
said survival is over a time period of 6 months to 3 years from said measurement of said plurality of respirations.

9. (canceled)

10. The method according to claim 1 comprising:
performing an olfactory test on the subject; and
identifying presence or lack of a sniff response in response to said olfactory test, from subject nasal airflow measurements including at least one measured sniff volume;
wherein said one or more respiration parameter comprises said presence or lack of a sniff response; and
wherein said determining is using said presence or lack of a sniff response.

11. (canceled)

12. The method of claim 10, wherein said performing comprises exposing said subject to an odor;
wherein said measuring comprises measuring nasal inhalation during said exposing to provide said at least one measured sniff volume and for a period of time not during said exposing to provide a volume of at least one baseline inhalation;
wherein said identifying comprises at least one of
comparing said sniff volume with said baseline inhalation; and
comparing a normalized sniff volume and a threshold, where the normalized sniff volume is a ratio between said sniff volume and a volume of at least one baseline inhalation, to identify said presence or lack of said sniff response.

13-14. (canceled)

15. The method according to claim 12 , wherein said measuring is for a period of time providing a volume of a plurality of baseline inhalations,
wherein said identifying comprises at least one of:
comparing said measured sniff volume with an average of said at least one baseline inhalation; and
excluding outlying baseline inhalations, from said at least one baseline inhalation, wherein said average of said at least one baseline inhalation is with included baseline inhalations.

16. The method of claim 15, wherein said period of time is immediately preceding said performing.

17. (canceled)

18. The method according to claim 10 , comprising:
repeating said performing and said identifying to provide a plurality of sniff volumes;
determining a sniff volume variability from said plurality of sniff volumes; and
comparing said sniff volume variability to a variability threshold to determine said presence or lack of a sniff response;
wherein a higher variability of respiration parameters indicates that the subject is in a higher state of consciousness and/or is more likely to recover and/or to survive.

19. The method according to claim 18, wherein said performing comprises one of:
-presenting a single scent to said subject a plurality of times; and
presenting a first scent, or presenting a second scent, or performing a blank presentation.

20-21. (canceled)

22. The method according to claim 10 , wherein a sniff volume is excluded from said identifying if said sniff volume exceeds a threshold.

23. The method of claim 10, comprising repeating said measuring, said performing and said identifying at intervals of time, to perform, over time, said determining .

24. The method of claim 23, wherein said intervals of time are 1 day to 1 month.

25-33. (canceled)

34. A method of assessing a subject comprising:
measuring subject respiration;
performing an olfactory test on said subject;
identifying baseline inhalations and sniffing inhalations;
excluding outlying baseline inhalations and outlying sniffing inhalations; and
assessing said subject based on included baseline inhalations and included sniffing inhalations.

35. The method of claim 34, wherein said assessing comprises assessing said subject using a normalized sniff volume, which is determined by normalizing said included sniffing inhalations using an averaged baseline inhalation determined from said included baseline inhalations.

36. The method of claim 35, comprising comparing said normalized sniff volume with a threshold to assess said subject.

37. The method according to claim 34 , comprising:
-repeating said performing, said identifying, said excluding and said assessing to provide a plurality of normalized sniff volumes;
excluding normalized sniff volumes of said plurality of normalized sniff volumes outside a threshold; and comparing a variability of included normalized sniff volumes with a sniff volume variability threshold to assess said subject.

38. (canceled)

39. The method of claim 1, wherein the subject is suffering from a brain injury.

40. The method of claim 1, wherein said determining one or more respiration parameter includes receiving a measurement signal from at least one sensor configured to sense respiration of the subject.
